SEQUENCE LISTING (10 sequences)

SEQ ID NO: 1 - protein, 210 aa, Artificial Sequence
  Synthetic construct
GLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 2 - protein, 173 aa, Artificial Sequence
  Synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 3 - protein, 42 aa, Artificial Sequence
  Synthetic construct
KRGRKKLLYIFKQPFMRPVQTTQEEDGCSCRFPEEEEGGCEL

SEQ ID NO: 4 - protein, 85 aa, Artificial  Sequence
  Synthetic construct
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 5 - protein, 21 aa, Artificial  Sequence
  Synthetic construct
VLLPLVIFFGLCLLSLLFIGL

SEQ ID NO: 6 - protein, 17 aa, Artificial  Sequence
  Synthetic construct
LGWLCLLLLPIPLIVWV

SEQ ID NO: 7 - protein, 273 aa, Artificial Sequence
  Synthetic construct
GLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLKRGRKKLLYIFKQPFMRPVQTTQEEDGCSCRFPEEEEGGCEL

SEQ ID NO: 8 - protein, 316 aa, Artificial  Sequence
  Synthetic construct
GLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 9 - protein, 232 aa, Artificial  Sequence
  Synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRGRKKLLYIFKQPFMRPVQTTQEEDGCSCRFPEEEEGGCEL

SEQ ID NO: 10 - protein, 275 aa, Artificial  Sequence
  Synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVSWRRRQRRLRGASSAEAPDGDKDAPEPLDKVIILSPGISDATAPAWPPPGEDPGTTPPGHSVPVPATELGSTELVTTKTAGPEQQ

(19) United States
(12) Patent Application Publication
(10) Pub. No.: US 2022/0348936 A1
(43) Pub. Date: Nov. 3, 2022

(54) CHIMERIC COSTIMULATORY RECEPTORS AND METHODS AND USES THEREOF

(71) Applicant: McMaster University, Hamilton (CA)

(72) Inventors: Jonathan Bramson, Oakville (CA); Phillip Marvyn, Brantford (CA)

(21) Appl. No.: 17/753,119

(22) PCT Filed: Sep. 16, 2020
(86) PCT No.: PCT/CA2020/051245

§ 371 Date: Feb. 18, 2022

Related U.S. Application Data
(60) Provisional application No. 62/900,911, filed on Sep. 16, 2019.

(51) Int. Cl. C12N 15/62; C07K 14/705; C07K 14/715; C12N 5/0783

(52) U.S. Cl. CPC C12N 15/62; C07K 14/70578; C07K 14/7151; C12N 5/0638; C07K 2319/02; C07K 2319/03

(57) ABSTRACT

Described herein is a chimeric costimulatory receptor (CCR) molecule having an extracellular domain of a Tumor Necrosis Factor Superfamily member, a transmembrane domain and a cytosolic costimulatory signaling domain from a Tumor Necrosis Factor Receptor Superfamily member is provided. Also provided are pharmaceutical compositions having a T cell expressing CCR and methods and uses of such T cells to treat cancers.

### RELATED APPLICATIONS

The present application claims the benefit of priority from U.S. Provisional Patent Application No. 62/900,911, filed on Sep. 16, 2019, the contents of which are incorporated by reference in their entirety.

### FIELD

The present application relates to chimeric receptors derived from the tumor-necrosis factor receptor superfamily. In particular, the application relates to chimeric tumor-necrosis factor receptor superfamily receptors that can co-stimulate immune cells, and their associated methods and uses for the treatment of cancer.

### BACKGROUND

Immune surveillance is the ability of the immune system to continually monitor the body for transformed cells. Cancer is able to circumvent this system and escape immune control. T cells are believed to be a major contributor to immune surveillance and cancer control. Over the last few decades therapies have been developed that attempt to harness the innate ability of T cells to respond to and eradicate tumors. The first approaches used autologous naturally occurring tumor-infiltrating lymphocytes that were expanded in vitro to obtain large numbers and reinfused into the patient. Improvements were made to these approaches by isolating T cells from peripheral blood mononuclear cells (PBMC's) and engineering them to express modified T cell receptors. Other approaches have focused on stimulating or redirecting T cells in vivo with targeted bispecific antibodies, with limited efficacy.

Cytotoxic T lymphocytes (CTLs) mediate the identification and clearance of transformed and/or virally infected cells through the T cell receptor (TCR), cytotoxic effector molecules and secreted cytokines. Natural TCR maturation undergoes multiple rounds of selection to recognize antigen through an MHC-dependent TCR interaction. MHC-TCR interaction initiates T cell activation and causes the release of effector molecules that act on tumor cells to sensitize and induce apoptosis. Cancerous cells often have impairments in the MHC peptide presentation machinery resulting in a downregulation of antigen presentation. The loss of antigen, and thus recognition of transformed cells, attenuates the adaptive immune response. The use of engineered T cells carrying non-MHC restricted synthetic antigen receptors offers a strategy to target tumors in which the adaptive immune system has not produced a robust enough response to clear the burden. Non-MHC restricted synthetic antigen receptors encompass a novel class of T cell activating receptors, which includes: Chimeric Antigen Receptors (CARs), Tri-functional Antigen Receptors (TACs), as well as other receptors capable of activating T cells following antigen binding. T cells engineered to express these novel synthetic antigen receptors acquire the ability to target a novel antigen, in addition to their native TCR, and mediate recognition and T cell activation upon tumor-antigen interaction. Interestingly, these receptors allow the ability to target epitopes beyond the natural TCR repertoire, expanding antigen selection to molecules such as carbohydrates and glycolipids. Engineered T cell therapy offers an attractive approach to cancer therapy because of its ability to target non-MHC restricted tumor antigens, ease of obtaining PBMC's, and increased safety over systemic administration of agonist antibodies. The clinical success of CAR-engineered T cells has established the feasibility and therapeutic potency of this novel class of cellular drug.

Costimulatory/coinhibitory signals are those that take place at the same time as TCR ligation and regulate T cell function. The type and strength of costimulatory/coinhibitory signals dictate the progression of the adaptive immune response. The Tumor-Necrosis Factor Receptor Superfamily (TNFRSF) and ligands are key costimulatory/coinhibitory molecules. Members of the TNFRSF mediate responses in the T-cell in multiple ways. Depending on the inflammatory milieu, signals propagated by TNFRSF can stimulate or inhibit the differentiation of effector to memory T cells generated from naïve T cells in response to antigen and during the memory response. T cells receive unique activation or survival signals at each stage of the response, including naive, effector, and memory stages; costimulatory signals are a main component driving these responses. Interestingly, costimulatory signals can be used to drive T cell responses while limiting coinhibitory signals. However, it is not fully understood how these responses are orchestrated. It has been shown that expression of several TNFRSFs are induced following T cell activation and that the expression of these molecules is non-ubiquitous suggesting that these molecules may have a role in modulating the immune response in different populations. Also, various stimuli can promote the expression of the TNFRSF ligands by antigen-presenting cells (APCs), including innate (bacterial ligands) and adaptive signals (proinflammatory cytokines). Therefore, TNFRSF serve to regulate T cell immunity through both expression of receptors on T cells and their ligand expression on other immune cells at each stage of the adaptive immune response.

### SUMMARY

The present inventors have demonstrated that a chimeric costimulatory receptor (CCR) comprised of functional domains from different Tumor-Necrosis Factor Receptor Superfamily (TNFRSF) receptors are able to reprogram the signaling caused by TNFRSF ligands to desired responses, including a T cell stimulation response.

Accordingly, disclosed herein, in certain embodiments, are Chimeric Costimulatory Receptor (CCR) nucleic acids, comprising (a) a first polynucleotide encoding an extracellular domain of a member of the Tumor Necrosis Factor Receptor Superfamily (TNFRSF); (b) a second polynucleotide encoding a transmembrane domain polypeptide; and (c) a third polynucleotide encoding a cytosolic costimulatory signaling domain polypeptide (e.g., from a Tumor Necrosis Factor Receptor Superfamily (TNFRSF) member). In some embodiments, the first polynucleotide and the third polynucleotide are derived from different members of the TNFRSF. In some embodiments, the first polynucleotide encodes an extracellular domain of a TNFRSF member having a death domain. In some embodiments, the first polynucleotide encodes an extracellular domain of Tumor necrosis factor receptor 1 (TNFR1), Tumor necrosis factor receptor 2 (TNFR2), Fas receptor, Death domain 4 (DR4), Death domain 5 (DR5), Death domain 3 (DR3), Death domain 6 (DR6), Ectodermal dysplasia receptor (EDAR), Ectodysplasin A2 receptor (XEDAR), TROY, or Nerve growth factor receptor (NGFR). In some embodiments, the first polynucleotide encodes an extracellular domain of TNFR1. In some embodiments, the first polynucleotide encodes an extracellular domain of TNFR2. In some embodiments, the first polynucleotide encodes an extracellular domain of Fas. In some embodiments, the first polynucleotide encodes an extracellular domain of DR4. In some embodiments, the first polynucleotide encodes an extracellular domain of DR5. In some embodiments, the first polynucleotide encodes an oligopeptide at least 80%, at least 85%, at least 90%, at least 95%, at least 99% or 100% identical to SEQ ID NO: 1. In some embodiments, the first polynucleotide encodes an oligopeptide at least 80%, at least 85%, at least 90%, at least 95%, at least 99% or 100% identical to SEQ ID NO: 2. In some embodiments, the third polynucleotide encodes a cytosolic costimulatory signaling domain from 4-1BB, BAFFR, OX40, CD27, CD40, GITR, HVEM, OX40, RELT, TACI, TROY, or TWEAK. In some embodiments, the third polynucleotide encodes a cytosolic costimulatory signaling domain from BAFFR. In some embodiments, the third polynucleotide encodes a cytosolic costimulatory signaling domain from 4-1BB. In some embodiments, the third polynucleotide encodes a cytosolic costimulatory signaling domain from CD27. In some embodiments, the third polynucleotide encodes a cytosolic costimulatory signaling domain from HVEM. In some embodiments, the third polynucleotide encodes a cytosolic costimulatory signaling domain from OX40. In some embodiments, the third polynucleotide encodes a cytosolic costimulatory signaling domain from GITR. In some embodiments, the third polynucleotide encodes a cytosolic costimulatory signaling domain from TACI. In some embodiments, the third polynucleotide encodes an oligopeptide at least 80%, at least 85%, at least 90%, at least 95%, at least 99% or 100% identical to SEQ ID NO: 3. In some embodiments, the third polynucleotide encodes an oligopeptide at least 80%, at least 85%, at least 90%, at least 95%, at least 99% or 100% identical to SEQ ID NO: 4. In some embodiments, the transmembrane domain polypeptide is the transmembrane domain polypeptide from a member of the Tumor Necrosis Factor Receptor Superfamily. In some embodiments, the second polynucleotide encodes an oligopeptide at least 80%, at least 85%, at least 90%, at least 95%, at least 99% or 100% identical to SEQ ID NO: 5. In some embodiments, the second polynucleotide encodes an oligopeptide at least 80%, at least 85%, at least 90%, at least 95%, at least 99% or 100% identical to SEQ ID NO: 6. In some embodiments, the transmembrane domain and cytosolic costimulatory signaling domain are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly and/or indirectly (e.g., via a linker) to the third polynucleotide. In some embodiments, the first polynucleotide and third polynucleotide are joined directly and/or indirectly (e.g., via a linker) to the second polynucleotide.

Further disclosed herein, in certain embodiments, are Chimeric Costimulatory Receptor (CCR) nucleic acids, comprising: (a) a first polynucleotide encoding an extracellular domain of Tumor Necrosis Factor Receptor 1; (b) a second polynucleotide encoding a transmembrane domain polypeptide; and (c) a third polynucleotide encoding a cytosolic costimulatory signaling domain polypeptide (e.g., from a Tumor Necrosis Factor Receptor Superfamily (TNFRSF) member). In some embodiments, the first polynucleotide encodes an oligopeptide at least 80%, at least 85%, at least 90%, at least 95%, at least 99% or 100% identical to SEQ ID NO: 1. In some embodiments, the cytosolic costimulatory signaling domain is a cytosolic costimulatory signaling domain from 4-1BB, BAFFR, OX40, CD27, CD40, GITR, HVEM, OX40, RELT, TACI, TROY, or TWEAK. In some embodiments, the cytosolic costimulatory signaling domain is a cytosolic costimulatory signaling domain from BAFFR. In some embodiments, the cytosolic costimulatory signaling domain is a cytosolic costimulatory signaling domain from 4-1BB. In some embodiments, the cytosolic costimulatory signaling domain is a cytosolic costimulatory signaling domain from CD27. In some embodiments, the cytosolic costimulatory signaling domain is a cytosolic costimulatory signaling domain from HVEM. In some embodiments, the cytosolic costimulatory signaling domain is a cytosolic costimulatory signaling domain from OX40. In some embodiments, the cytosolic costimulatory signaling domain is a cytosolic costimulatory signaling domain from GITR. In some embodiments, the cytosolic costimulatory signaling domain is a cytosolic costimulatory signaling domain from TACI. In some embodiments, the third polynucleotide encodes an oligopeptide at least 80%, at least 85%, at least 90%, at least 95%, at least 99% or 100% identical to SEQ ID NO: 3. In some embodiments, the third polynucleotide encodes an oligopeptide at least 80%, at least 85%, at least 90%, at least 95%, at least 99% or 100% identical to SEQ ID NO: 4. In some embodiments, the transmembrane domain polypeptide is the transmembrane domain polypeptide from a member of the Tumor Necrosis Factor Receptor Superfamily. In some embodiments, the second polynucleotide encodes an oligopeptide at least 80%, at least 85%, at least 90%, at least 95%, at least 99% or 100% identical to SEQ ID NO: 5. In some embodiments, the second polynucleotide encodes an oligopeptide at least 80%, at least 85%, at least 90%, at least 95%, at least 99% or 100% identical to SEQ ID NO: 6. In some embodiments, the transmembrane domain and cytosolic costimulatory signaling domains are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly and/or indirectly (e.g., via a linker) to the third polynucleotide. In some embodiments, the first polynucleotide and third polynucleotide are joined directly or indirectly (e.g., via a linker) to the second polynucleotide.

Further disclosed herein, in certain embodiments, are Chimeric Costimulatory Receptor (CCR) nucleic acids, comprising: (a) a first polynucleotide encoding an extracellular domain of Tumor Necrosis Factor Receptor 2; (b) a second polynucleotide encoding a transmembrane domain polypeptide; and (c) a third polynucleotide encoding a cytosolic costimulatory signaling domain polypeptide (e.g., from a Tumor Necrosis Factor Receptor Superfamily (TNFRSF) member). In some embodiments, the cytosolic costimulatory signaling domain is a cytosolic costimulatory signaling domain from 4-1BB, BAFFR, OX40, CD27, CD40, GITR, HVEM, OX40, RELT, TACI, TROY, or TWEAK. In some embodiments, the cytosolic costimulatory signaling domain is a cytosolic costimulatory signaling domain from BAFFR. In some embodiments, the cytosolic costimulatory signaling domain is a cytosolic costimulatory signaling domain from 4-1BB. In some embodiments, the third polynucleotide encodes a cytosolic costimulatory signaling domain from CD27. In some embodiments, the third polynucleotide encodes a cytosolic costimulatory signaling domain from HVEM. In some embodiments, the third polynucleotide encodes a cytosolic costimulatory signaling domain from OX40. In some embodiments, the third polynucleotide encodes a cytosolic costimulatory signaling domain from GITR. In some embodiments, the third polynucleotide encodes a cytosolic costimulatory signaling domain from TACI. In some embodiments, the third polynucleotide encodes an oligopeptide at least 80%, at least 85%, at least 90%, at least 95%, at least 99% or 100% identical to SEQ ID NO: 3. In some embodiments, the third polynucleotide encodes an oligopeptide at least 80%, at least 85%, at least 90%, at least 95%, at least 99% or 100% identical to SEQ ID NO: 4. In some embodiments, the transmembrane domain polypeptide is the transmembrane domain polypeptide from a member of the Tumor Necrosis Factor Receptor Superfamily. In some embodiments, the second polynucleotide encodes an oligopeptide at least 80%, at least 85%, at least 90%, at least 95%, at least 99% or 100% identical to SEQ ID NO: 5. In some embodiments, the second polynucleotide encodes an oligopeptide at least 80%, at least 85%, at least 90%, at least 95%, at least 99% or 100% identical to SEQ ID NO: 6. In some embodiments, the transmembrane domain and cytosolic costimulatory signaling domain are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly and/or indirectly (e.g., via a linker) to the third polynucleotide. In some embodiments, the first polynucleotide and third polynucleotide are joined directly and/or indirectly (e.g., via a linker) to the second polynucleotide.

Further disclosed herein, in certain embodiments, are Chimeric Costimulatory Receptor (CCR) nucleic acids, comprising: (a) a first polynucleotide encoding an extracellular domain of Tumor Necrosis Factor Receptor Superfamily Member 6 (TNFRSF6; Fas); (b) a second polynucleotide encoding a transmembrane domain polypeptide; and (c) a third polynucleotide encoding a cytosolic costimulatory signaling domain polypeptide (e.g., from a Tumor Necrosis Factor Receptor Superfamily (TNFRSF) member). In some embodiments, the first polynucleotide encodes an oligopeptide at least 80%, at least 85%, at least 90%, at least 95%, at least 99% or 100% identical to SEQ ID NO: 2. In some embodiments, the cytosolic costimulatory signaling domain is a cytosolic costimulatory signaling domain from 4-1BB, BAFFR, OX40, CD27, CD40, GITR, HVEM, OX40, RELT, TACI, TROY, or TWEAK. In some embodiments, the cytosolic costimulatory signaling domain is a cytosolic costimulatory signaling domain from BAFFR. In some embodiments, the cytosolic costimulatory signaling domain is a cytosolic costimulatory signaling domain from 4-1BB. In some embodiments, the third polynucleotide encodes a cytosolic costimulatory signaling domain from CD27. In some embodiments, the third polynucleotide encodes a cytosolic costimulatory signaling domain from HVEM. In some embodiments, the third polynucleotide encodes a cytosolic costimulatory signaling domain from OX40. In some embodiments, the third polynucleotide encodes a cytosolic costimulatory signaling domain from GITR. In some embodiments, the third polynucleotide encodes a cytosolic costimulatory signaling domain from TACI. In some embodiments, the third polynucleotide encodes an oligopeptide at least 80%, at least 85%, at least 90%, at least 95%, at least 99% or 100% identical to SEQ ID NO: 3. In some embodiments, the third polynucleotide encodes an oligopeptide at least 80%, at least 85%, at least 90%, at least 95%, at least 99% or 100% identical to SEQ ID NO: 4. In some embodiments, the transmembrane domain polypeptide is the transmembrane domain polypeptide from a member of the Tumor Necrosis Factor Receptor Superfamily. In some embodiments, the second polynucleotide encodes an oligopeptide at least 80%, at least 85%, at least 90%, at least 95%, at least 99% or 100% identical to SEQ ID NO: 5. In some embodiments, the second polynucleotide encodes an oligopeptide at least 80%, at least 85%, at least 90%, at least 95%, at least 99% or 100% identical to SEQ ID NO: 6. In some embodiments, the transmembrane domain and cytosolic costimulatory signaling domains are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly and/or indirectly (e.g., via a linker) to the third polynucleotide. In some embodiments, the first polynucleotide and third polynucleotide are joined directly and/or indirectly (e.g., via a linker) to the second polynucleotide.

Further disclosed herein, in certain embodiments, are polypeptides encoded by a Chimeric Costimulatory Receptor (CCR) nucleic acid described herein.

Further disclosed herein, in certain embodiments, are Chimeric Costimulatory Receptor (CCR) polypeptides at least 80%, at least 85%, at least 90%, at least 95%, at least 99% or 100% identical to SEQ ID NO: 7.

Further disclosed herein, in certain embodiments, are Chimeric Costimulatory Receptor (CCR) polypeptides at least 80%, at least 85%, at least 90%, at least 95%, at least 99% or 100% identical to SEQ ID NO: 8.

Further disclosed herein, in certain embodiments, are Chimeric Costimulatory Receptor (CCR) polypeptides at least 80%, at least 85%, at least 90%, at least 95%, at least 99% or 100% identical to SEQ ID NO: 9.

Further disclosed herein, in certain embodiments, are Chimeric Costimulatory Receptor (CCR) polypeptides at least 80%, at least 85%, at least 90%, at least 95%, at least 99% or 100% identical to SEQ ID NO: 10.

Additionally, disclosed herein, in certain embodiments, are T-cells expressing a Chimeric Costimulatory Receptor (CCR) nucleic acid described herein, or comprising a polypeptide disclosed herein. In some embodiments, the T cell is a cytotoxic T cell, helper T cell, regulatory T cell, or gamma-delta T cell.

Also disclosed herein, in certain embodiments, are T cells (e.g., for treating a cancer in a subject in need thereof), comprising (a) a Chimeric Costimulatory Receptor (CCR) nucleic acid disclosed herein; and (b) a second nucleic acid encoding an engineered T cell receptor (TCR) or a synthetic antigen receptor polypeptide that can recognize a target-specific ligand. In some embodiments, the target-specific ligand binds an antigen on a cancerous cell. In some embodiments, the synthetic antigen receptor polynucleotide is a Chimeric Antigen Receptor (CAR), a T cell Antigen Coupler (TAC), or a BiTE (Bispecific T-cell Engager). In some embodiments, the synthetic antigen receptor polynucleotide is a T cell Antigen Coupler (TAC). In some embodiments, the T cell is a cytotoxic T cell, helper T cell, regulatory T cell, or gamma-delta T cell.

Also disclosed herein, in certain embodiments, are T cells (e.g., for treating a cancer in a subject in need thereof), comprising (a) a Chimeric Costimulatory Receptor (CCR) nucleic acid comprising: (i) a first polynucleotide encoding an extracellular domain from Tumor Necrosis Factor Receptor 1; (ii) a second polynucleotide encoding a transmembrane domain polypeptide; and (iii) a third polynucleotide encoding a cytosolic costimulatory signaling domain polypeptide (e.g., from a Tumor Necrosis Factor Receptor Superfamily (TNFRSF) member); and (b) a second nucleic acid encoding an engineered T cell receptor (TCR) or a synthetic antigen receptor polypeptide that can recognize a target-specific ligand. In some embodiments, the third polynucleotide of the Chimeric Costimulatory Receptor (CCR) nucleic acid encodes a cytosolic costimulatory signaling domain from 4-1BB, BAFFR, OX40, CD27, CD40, GITR, HVEM, OX40, RELT, TACI, TROY, or TWEAK. In some embodiments, the third polynucleotide of the Chimeric Costimulatory Receptor (CCR) nucleic acid encodes a cytosolic costimulatory signaling domain from BAFFR. In some embodiments, the third polynucleotide of the Chimeric Costimulatory Receptor (CCR) nucleic acid encodes a cytosolic costimulatory signaling domain from 4-1BB. In some embodiments, the third polynucleotide encodes a cytosolic costimulatory signaling domain from CD27. In some embodiments, the third polynucleotide encodes a cytosolic costimulatory signaling domain from HVEM. In some embodiments, the third polynucleotide encodes a cytosolic costimulatory signaling domain from OX40. In some embodiments, the third polynucleotide encodes a cytosolic costimulatory signaling domain from GITR. In some embodiments, the third polynucleotide encodes a cytosolic costimulatory signaling domain from TACI. In some embodiments, the transmembrane domain and cytosolic costimulatory signaling domain are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly or indirectly (e.g., via a linker) to the third polynucleotide. In some embodiments, the first polynucleotide and third polynucleotide are joined directly or indirectly (e.g., via a linker) to the second polynucleotide. In some embodiments, the target-specific ligand binds an antigen on a cancerous cell. In some embodiments, the synthetic antigen receptor polynucleotide is a Chimeric Antigen Receptor (CAR), a T cell Antigen Coupler (TAC), or a BiTE (Bispecific T-cell Engager). In some embodiments, the synthetic antigen receptor polynucleotide is a T cell Antigen Coupler (TAC). In some embodiments, the T cell is a cytotoxic T cell, helper T cell, regulatory T cell, or gamma-delta T cell.

Also disclosed herein, in certain embodiments, are T cells (e.g., for treating a cancer in a subject in need thereof), comprising (a) a Chimeric Costimulatory Receptor (CCR) nucleic acid comprising: (i) a first polynucleotide encoding an extracellular domain from Tumor Necrosis Factor Receptor 2; (ii) a second polynucleotide encoding a transmembrane domain polypeptide; and (iii) a third polynucleotide encoding a cytosolic costimulatory signaling domain polypeptide (e.g., from a Tumor Necrosis Factor Receptor Superfamily (TNFRSF) member); and (b) a second nucleic acid encoding an engineered T cell receptor (TCR) or a synthetic antigen receptor polypeptide that can recognize a target-specific ligand. In some embodiments, the third polynucleotide of the Chimeric Costimulatory Receptor (CCR) nucleic acid encodes a cytosolic costimulatory signaling domain from 4-1BB, BAFFR, OX40, CD27, CD40, GITR, HVEM, OX40, RELT, TACI, TROY, or TWEAK. In some embodiments, the third polynucleotide of the Chimeric Costimulatory Receptor (CCR) nucleic acid encodes a cytosolic costimulatory signaling domain from BAFFR. In some embodiments, the third polynucleotide of the Chimeric Costimulatory Receptor (CCR) nucleic acid encodes a cytosolic costimulatory signaling domain from 4-1BB. In some embodiments, the third polynucleotide of the Chimeric Costimulatory Receptor (CCR) nucleic acid encodes a cytosolic costimulatory signaling domain from CD27. In some embodiments, the third polynucleotide of the Chimeric Costimulatory Receptor (CCR) nucleic acid encodes a cytosolic costimulatory signaling domain from HVEM. In some embodiments, the third polynucleotide of the Chimeric Costimulatory Receptor (CCR) nucleic acid encodes a cytosolic costimulatory signaling domain from OX40. In some embodiments, the third polynucleotide of the Chimeric Costimulatory Receptor (CCR) nucleic acid encodes a cytosolic costimulatory signaling domain from GITR. In some embodiments, the third polynucleotide of the Chimeric Costimulatory Receptor (CCR) nucleic acid encodes a cytosolic costimulatory signaling domain from TACI. In some embodiments, the transmembrane domain and cytosolic costimulatory signaling domain are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly or indirectly (e.g., via a linker) to the third polynucleotide. In some embodiments, the first polynucleotide and third polynucleotide are joined directly or indirectly (e.g., via a linker) to the second polynucleotide. In some embodiments, the target-specific ligand binds an antigen on a cancerous cell. In some embodiments, the synthetic antigen receptor polynucleotide is a Chimeric Antigen Receptor (CAR), a T cell Antigen Coupler (TAC), or a BiTE (Bispecific T-cell Engager). In some embodiments, the synthetic antigen receptor polynucleotide is a T cell Antigen Coupler (TAC). In some embodiments, the T cell is a cytotoxic T cell, helper T cell, regulatory T cell, or gamma-delta T cell.

Also disclosed herein, in certain embodiments, are T cells (e.g., for treating a cancer in a subject in need thereof), comprising (a) a Chimeric Costimulatory Receptor (CCR) nucleic acid comprising: (i) a first polynucleotide encoding an extracellular domain from Fas receptor; (ii) a second polynucleotide encoding a transmembrane domain polypeptide; and (iii) a third polynucleotide encoding a cytosolic costimulatory signaling domain polypeptide (e.g., from a Tumor Necrosis Factor Receptor Superfamily (TNFRSF) member); and (b) a second nucleic acid encoding a an engineered T cell receptor (TCR) or a synthetic antigen receptor polypeptide that can recognize a target-specific ligand. In some embodiments, the third polynucleotide of the Chimeric Costimulatory Receptor (CCR) nucleic acid encodes a cytosolic costimulatory signaling domain from 4-1BB, BAFFR, OX40, CD27, CD40, GITR, HVEM, OX40, RELT, TACI, TROY, or TWEAK. In some embodiments, the third polynucleotide of the Chimeric Costimulatory Receptor (CCR) nucleic acid encodes a cytosolic costimulatory signaling domain from BAFFR. In some embodiments, the third polynucleotide of the Chimeric Costimulatory Receptor (CCR) nucleic acid encodes a cytosolic costimulatory signaling domain from 4-1BB. In some embodiments, the third polynucleotide of the Chimeric Costimulatory Receptor (CCR) nucleic acid encodes a cytosolic costimulatory signaling domain from CD27. In some embodiments, the third polynucleotide of the Chimeric Costimulatory Receptor (CCR) nucleic acid encodes a cytosolic costimulatory signaling domain from HVEM. In some embodiments, the third polynucleotide of the Chimeric Costimulatory Receptor (CCR) nucleic acid encodes a cytosolic costimulatory signaling domain from OX40. In some embodiments, the third polynucleotide of the Chimeric Costimulatory Receptor (CCR) nucleic acid encodes a cytosolic costimulatory signaling domain from GITR. In some embodiments, the third polynucleotide of the Chimeric Costimulatory Receptor (CCR) nucleic acid encodes a cytosolic costimulatory signaling domain from TACI. In some embodiments, the transmembrane domain and cytosolic costimulatory signaling domain are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly or indirectly (e.g., via a linker) to the third polynucleotide. In some embodiments, the first polynucleotide and third polynucleotide are joined directly or indirectly (e.g., via a linker) to the second polynucleotide. In some embodiments, the target-specific ligand binds an antigen on a cancerous cell. In some embodiments, the synthetic antigen receptor polynucleotide is a Chimeric Antigen Receptor (CAR), a T cell Antigen Coupler (TAC), or a BiTE (Bispecific T-cell Engager). In some embodiments, the synthetic antigen receptor polynucleotide is a T cell Antigen Coupler (TAC). In some embodiments, the T cell is a cytotoxic T cell, helper T cell, regulatory T cell, or gamma-delta T cell.

Disclosed herein, in certain embodiments, are methods of treating a cancer in an individual in need thereof, comprising administering to the individual an immune cell comprising Chimeric Costimulatory Receptor (CCR) nucleic acid, comprising (a) a first polynucleotide encoding an extracellular domain of a member of the Tumor Necrosis Factor Receptor Superfamily; (b) a second polynucleotide encoding a transmembrane domain polypeptide; and (c) a third polynucleotide encoding a cytosolic costimulatory signaling domain polypeptide (e.g., from a Tumor Necrosis Factor Receptor Superfamily (TNFRSF) member). In some embodiments, the first polynucleotide and the third polynucleotide are derived from different members of the TNFRSF. In some embodiments, the first polynucleotide encodes an extracellular domain of TNFRSF member having a death domain. In some embodiments, the first polynucleotide encodes an extracellular domain of TNFR1, TNFR2, Fas, DR4, DR5, DR3, DR6, EDAR, XEDAR, TROY or NGFR. In some embodiments, the third polynucleotide encodes a cytosolic costimulatory signaling domain polypeptide from 4-1BB, BAFFR, OX40, CD27, CD40, GITR, HVEM, OX40, RELT, TACI, TROY, or TWEAK. In some embodiments, the immune cell is a T cell. In some embodiments, the T cell is an engineered T cell. In some embodiments, the immune cell is a natural killer cell (NK cell). In some embodiments, the immune cell is a macrophage. In some embodiments, the immune cell is a tumor-infiltrating lymphocyte (TIL). In some embodiments, the immune cell is a monocyte. In some embodiments, the immune cell is a B cell. In some embodiments, the immune cell is an immune cell disclosed herein. In some embodiments, the cancer is a leukemia or lymphoma. In some embodiments, the cancer is mixed lineage leukemia (MLL), chronic lymphocytic leukemia (CLL), acute lymphoblastic leukemia (ALL), large B-cell lymphoma, diffuse large B-cell lymphoma, primary mediastinal B cell lymphoma, high grade B-cell lymphoma, or large B cell lymphoma arising from follicular lymphoma. In some embodiments, the cancer is a lung cancer, a breast cancer, a colon cancer, multiple myeloma, glioblastoma, gastric cancer, ovarian cancer, stomach cancer, colorectal cancer, urothelial cancer, endometrial cancer, or a melanoma. In some embodiments, the cancer is a lung cancer. In some embodiments, the cancer is a breast cancer. In some embodiments, the cancer is a colon cancer. In some embodiments, the cancer is multiple myeloma. In some embodiments, the cancer is a glioblastoma. In some embodiments, the cancer is a gastric cancer. In some embodiments, the cancer is an ovarian cancer. In some embodiments, the cancer is a stomach cancer. In some embodiments, the cancer is urothelial cancer. In some embodiments, the cancer is an endometrial cancer. In some embodiments, the cancer is a melanoma.

Disclosed herein, in certain embodiments, are pharmaceutical compositions comprising (a) an immune cell comprising (i) a first polynucleotide encoding an extracellular domain of a member of the Tumor Necrosis Factor Receptor Superfamily; (ii) a second polynucleotide encoding a transmembrane domain polypeptide; and (iii) a third polynucleotide encoding a cytosolic costimulatory signaling domain polypeptide (e.g., from a Tumor Necrosis Factor Receptor Superfamily (TNFRSF) member); and (b) a pharmaceutically acceptable carrier. In some embodiments, the first polynucleotide and the third polynucleotide are derived from different members of the TNFRSF. In some embodiments, the first polynucleotide encodes an extracellular domain of TNFRSF member having a death domain. In some embodiments, the first polynucleotide encodes an extracellular domain of TNFR1, TNFR2, Fas, DR4, DR5, DR3, DR6, EDAR, XEDAR, TROY or NGFR. In some embodiments, the first polynucleotide encodes an extracellular domain of TNFR1. In some embodiments, the first polynucleotide encodes an extracellular domain of TNFR2. In some embodiments, the first polynucleotide encodes an extracellular domain of Fas. In some embodiments, the third polynucleotide encodes a cytosolic costimulatory signaling domain from 4-1BB, BAFFR, OX40, CD27, CD40, GITR, HVEM, OX40, RELT, TACI, TROY, or TWEAK. In some embodiments, the third polynucleotide encodes a cytosolic costimulatory signaling domain from BAFFR. In some embodiments, the third polynucleotide encodes a cytosolic costimulatory signaling domain from 4-1BB. In some embodiments, the third polynucleotide encodes a cytosolic costimulatory signaling domain from CD27. In some embodiments, the third polynucleotide encodes a cytosolic costimulatory signaling domain from HVEM. In some embodiments, the third polynucleotide encodes a cytosolic costimulatory signaling domain from OX40. In some embodiments, the third polynucleotide encodes a cytosolic costimulatory signaling domain from GITR. In some embodiments, the third polynucleotide encodes a cytosolic costimulatory signaling domain from TACI. In some embodiments, the transmembrane domain and cytosolic costimulatory signaling domain are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly or indirectly (e.g., via a linker) to the third polynucleotide. In some embodiments, the first polynucleotide and third polynucleotide are joined directly or indirectly (e.g., via a linker) to the second polynucleotide. In some embodiments, the immune cell is a T cell, a natural killer cell (NK cell), a macrophage, a tumor-infiltrating lymphocyte (TIL), a monocyte, or a B cell. In some embodiments, the immune cell is an immune cell disclosed herein.

Disclosed herein, in certain embodiments, are vector constructs comprising: (a) a Chimeric Costimulatory Receptor (CCR) disclosed herein; and (b) a promoter functional in a mammalian cell.

Disclosed herein, in certain embodiments, are isolated or engineered T lymphocytes, natural killer cells, macrophages, tumor-infiltrating lymphocytes or monocytes transfected with a vector construct disclosed herein.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1: Principle of a TNFR1-4-1BB Chimeric Costimulatory Receptor.

Illustration of the hypothetical biology of a TNFR1-4-1BB Chimeric Costimulatory Receptor (CCR) in the context of the native T cell receptor (TCR). TNFα secreted by the activated T cell following ligation of the TCR leads to production of TNFα, which will ligate the CCR and promote activation of downstream pathways that lead to enhanced survival.

FIGS. 2A-B: Chimeric Costimulatory Receptor (CCR) Design.

FIG. 2A compares the full length native TNFR1 receptor (a), the TNF-Blocker receptor containing both the extracellular and transmembrane domains of native TNFR1 with a truncated non-signaling cytoplasmic domain (b), and a chimeric costimulatory receptor containing the extracellular and transmembrane domains of TNFR1 joined to the cytoplasmic costimulatory signaling domain of 4-1BB (c).

FIG. 2B compares the full length native Fas receptor (a), the Fas-TRUNC receptor containing both the extracellular and transmembrane domains of native Fas with a truncated non-signalling cytoplasmic domain (b), and a Fas-Chimera containing the extracellular and transmembrane domains of Fas joined to the cytoplasmic costimulatory signalling domain of 4-1BB or BAFFR (c).

FIG. 3: TNFR1-4-1BB enhances expression of NFκB promoters.

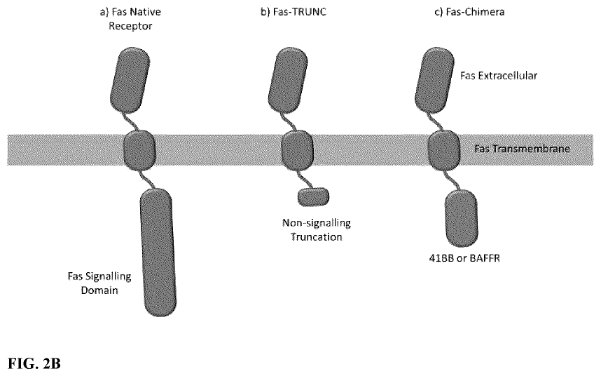

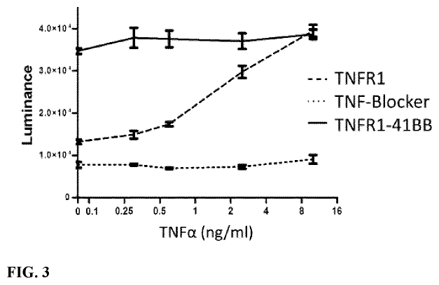

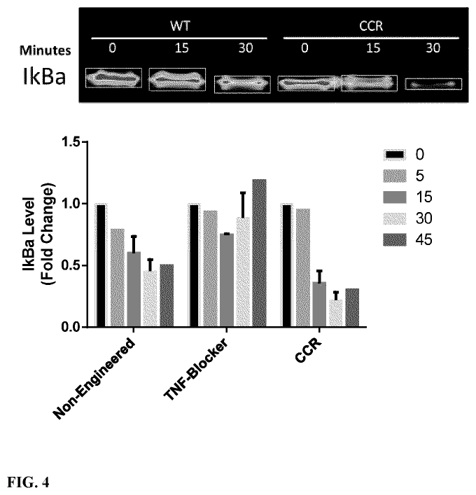

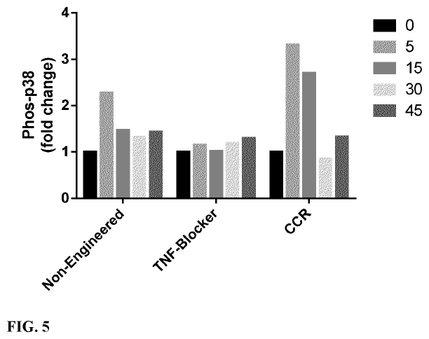

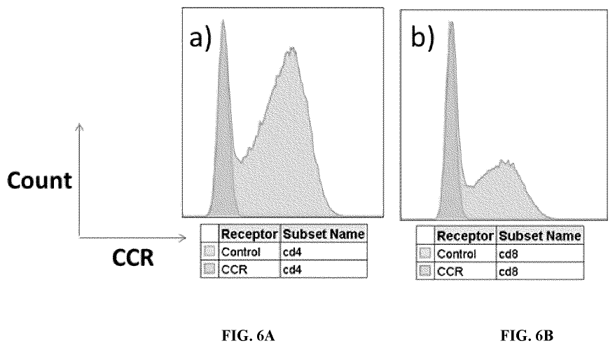

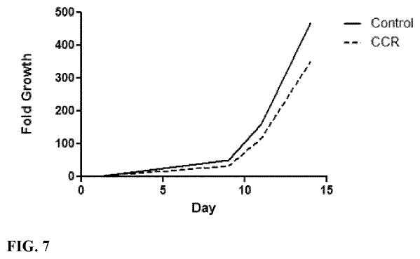

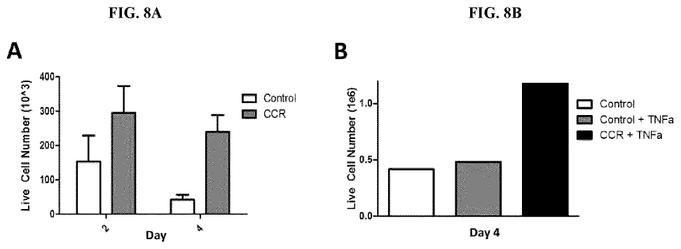

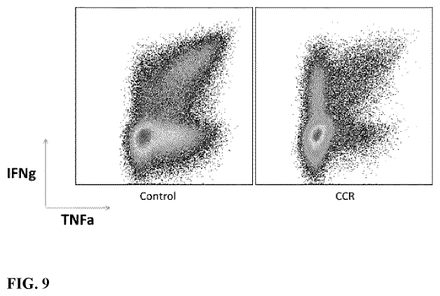

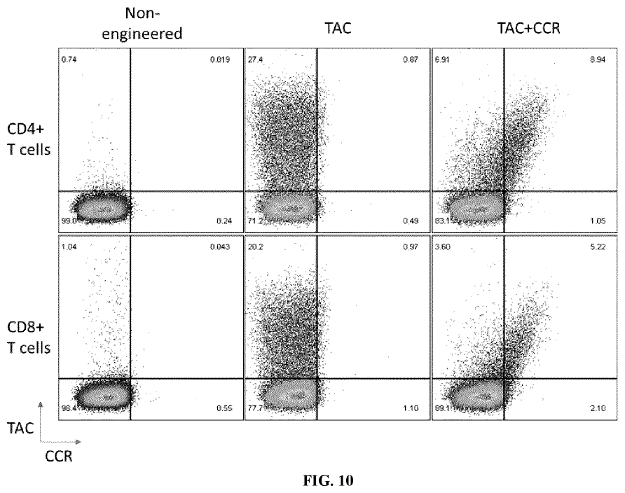

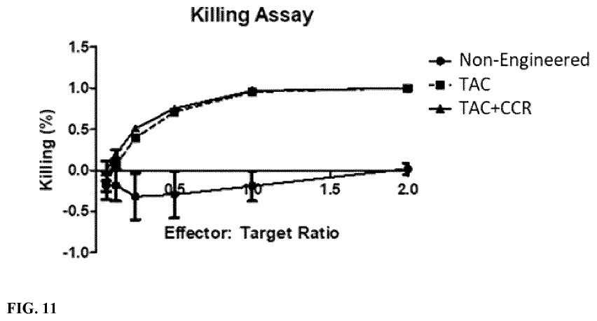

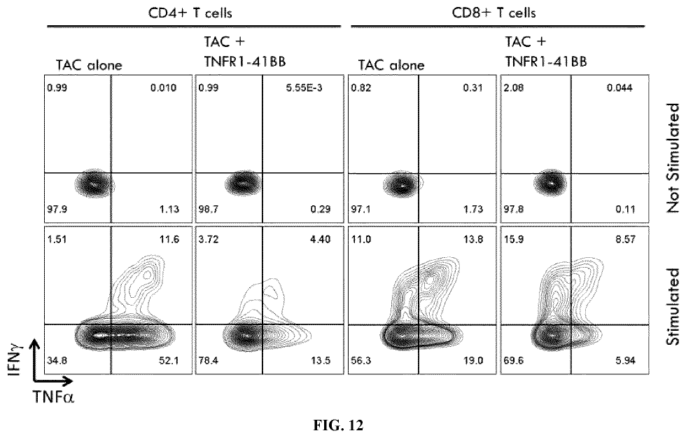

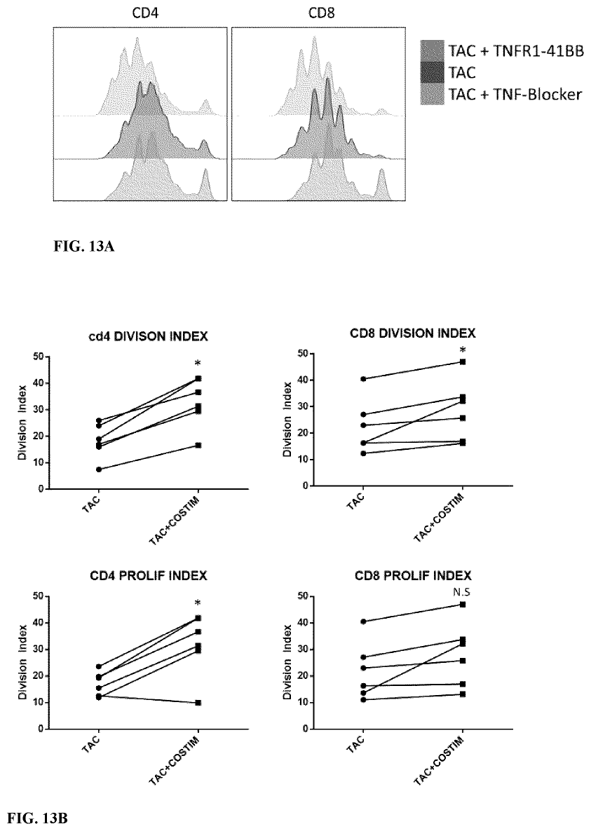

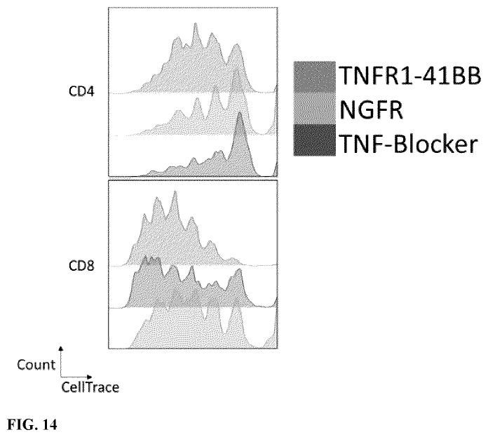

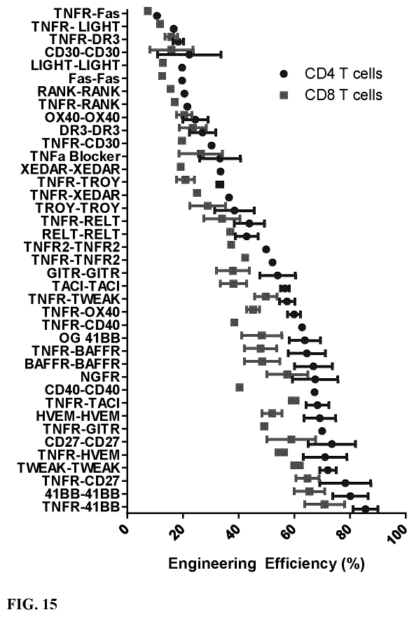

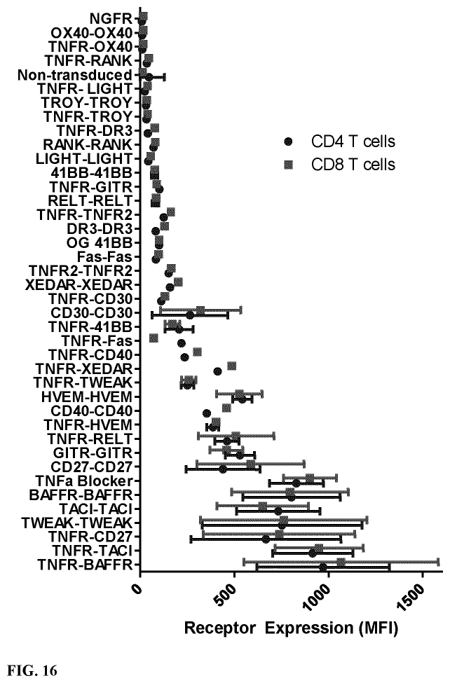

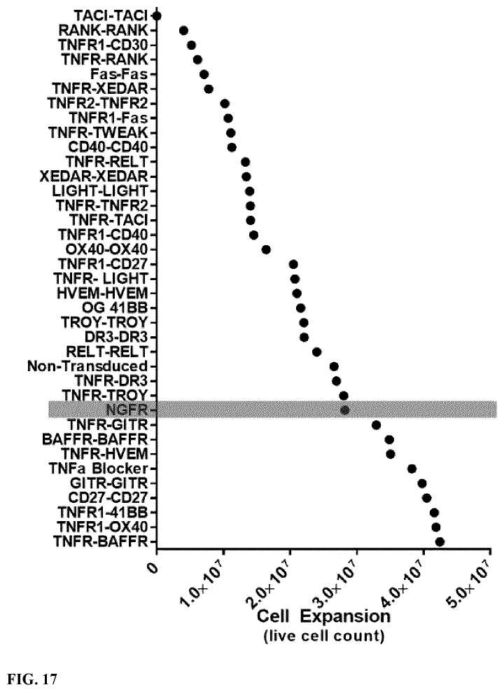

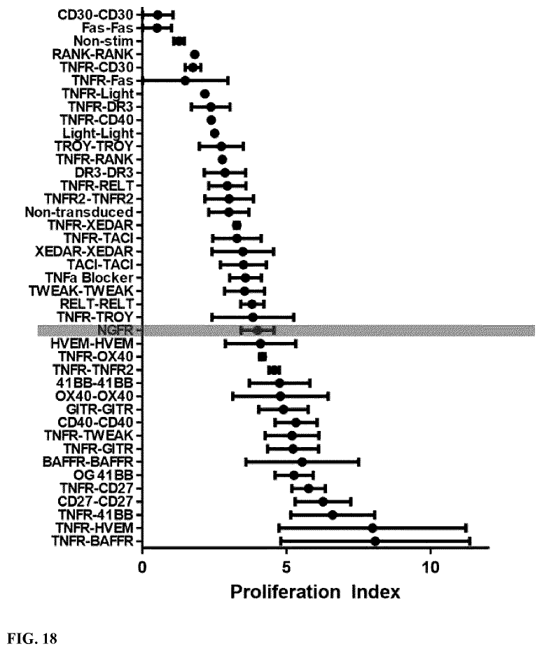

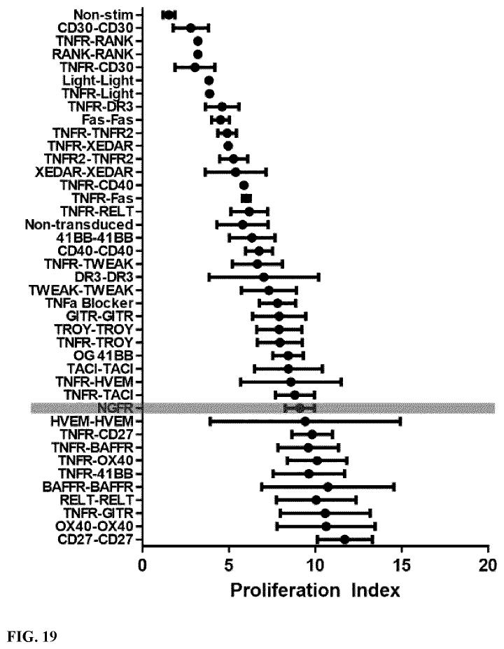

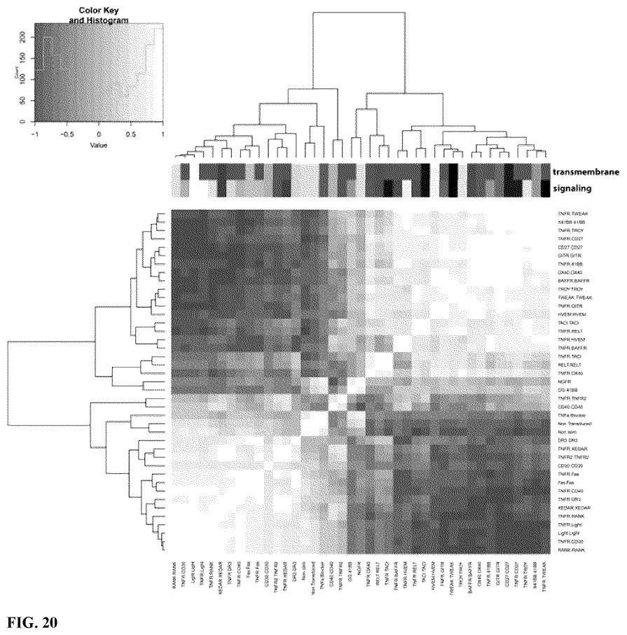

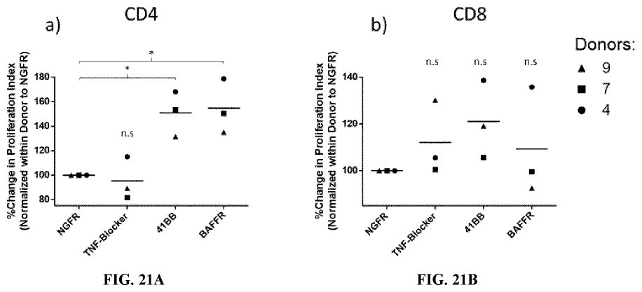

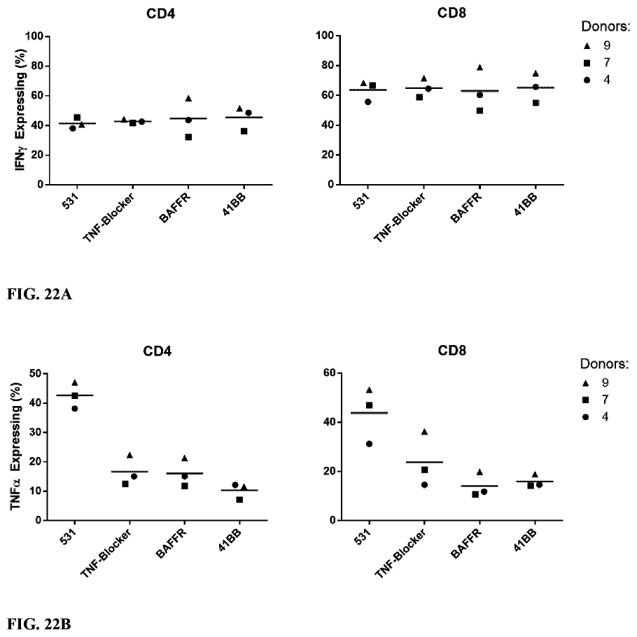

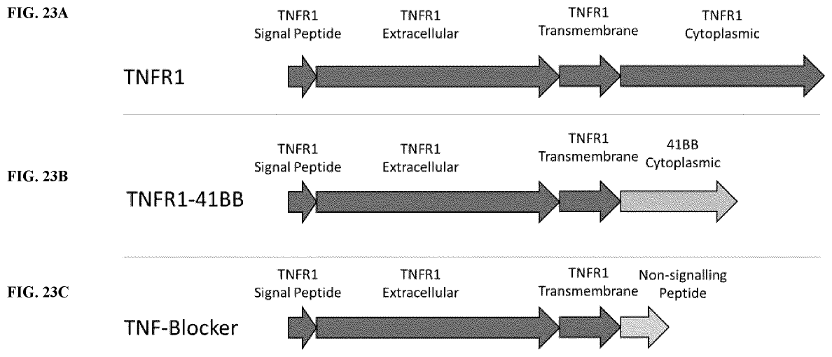

TNFR-fusion receptors signaling activity was evaluated using a luciferase reporter gene under the control of 3×NFkB enhancer elements. TNFR-fusion receptors from FIG. 2A were introduced separately into a HEK293TM cell line along with the NFkB-Luciferase reporter plasmid. Luciferase activity was measured under increasing concentrations of TNFα, the ligand for the TNFR-fusion receptors. HEK293TM cells carrying the native TNFR1 receptor demonstrated a dose-dependent increase in NFkB reporter activity. The TNF-Blocker receptor demonstrated abrogated reporter activity in response to increasing TNFα ligand. The TNFR1-4-1BB CCR demonstrated enhanced NFkB reporter activity at all concentrations of TNFα.

FIG. 4: Time-dependent degradation of IκBα upon CCR stimulation

Degradation of IκBα leads to the release of active NFκB. The Jurkat cell line was transduced to express the TNFR1-4-1BB CCR or TNF-Blocker receptor containing no signaling domain (see FIG. 2A). Jurkat cells were stimulated with 20 ng/ml TNFα for 0, 5, 15, 30, and 45 mins. Jurkat cells engineered with CCR have increased degradation of IkBα compared to non-engineered wildtype cells. The TNF-Blocker receptor abrogated IκBα degradation at all timepoints evaluated. (n=4)

FIG. 5: Time-dependent Phosphorylation of p38

Phosphorylation and activation of p38 MAPK leads to the upregulation of p38 genes. The Jurkat cell line was transduced to express a CCR or TNF-Blocker receptor containing no signaling domain (see FIG. 2A). Jurkat cells were stimulated with 20 ng/ml TNFα for 0, 5, 15, 30, and 45 mins. Jurkats cells engineered with CCR have increased phosphorylation of p38 compared to non-engineered wildtype cells. The TNF-Blocker receptor abrogated prevented p38 phosphorylation at all timepoints evaluated (n=1).

FIGS. 6A-B: Surface expression of the TNFR1-4-1BB CCR in primary human T cell subsets

Human PBMCs stimulated with anti-CD3/anti-CD28 beads and grown in IL2 and IL7 were engineered with the TNFR1-4-1BB CCR. On day 14 of culture engineered cells were stained for the CCR and assessed by flow cytometry. FIG. 6A represents CD4+ T cells; FIG. 6B represents CD8+ T cells.

FIG. 7: Growth rates of TNFR1-4-1BB CCR transduced primary human T cells in vitro

Human PBMCs stimulated with anti-CD3/anti-CD28 beads and grown in IL2 and IL7 were engineered with the TNFR1-4-1BB CCR. Live cell counts were recorded over the 14 day culture period.

FIGS. 8A-B: Survival of T cells expressing the the TNFR1-4-1BB CCR following cytokine withdrawal

FIG. 8A shows T cells stimulated with anti-CD3 alone without supplemented growth factors. FIG. 8B shows T cells stimulated with TNFα alone without supplemented cytokines.

FIG. 9: TNFR1-4-1BB CCR stimulation alters cytokine profile

T cells engineered with the the TNFR1-4-1BB CCR were tested on day 14 of culture for cytokine production following anti-CD3 stimulation. Cells were incubated on anti-CD3 coated plates for 4 hrs in the presence of a Golgi transport inhibitor (GolgiPlug®). Cells were stained and assessed by flow Cytometry for intracellular cytokine production.

FIG. 10: Phenotype of TAC+CCR T cells engineered with the 2A expression system

T cells were activated on day 0 with anti-CD3/anti-CD28 beads. One day later, the T cells were transduced with either a lentivirus carrying a TAC receptor directed against the myeloma protein, BCMA, or a lentivirus encoding the same TAC receptor along with the TNFR1-4-1BB CCR separated by a picornavirus 2A sequence (TAC+CCR). Cells were kept in culture with IL2 and IL7 with fresh media added every 2 days. Staining and detection of surface TAC and CCR by flow Cytometry were carried out on day 14 of culture. Surface protein levels of TAC in the 2A system were lower than the single expression system. The TNFR1-4-1BB CCR protein was detected on the surface of 2A engineered T cells.

FIG. 11: TAC+CCR T cells demonstrate lysis of BCMA+Tumor cell target

T cell mediated lysis of in-vitro tumor targets. T cells were co-incubated with KMS11 tumor targets, expressing luciferase enzyme, for 24 hrs. Luciferase activity was used as a measure of tumor cell lysis. UT (Non-Engineered) T cells demonstrated non detectable tumor lysis of KMS11 tumor targets. T cells engineered with TAC or TAC+CCR as described in FIG. 10. Both engineered T cell populations demonstrated similar lysis of tumor targets over a 24 hr assay.

FIG. 12: CCR engineered TAC T cells produce less inflammatory cytokines upon stimulation

Engineered T cells were coincubated with KMS11 tumor target in the presence of a Golgi transport inhibitor (Golgi Plug). Following a 4 hour coincubation, cells were stained for intracellular cytokine production. Cytokines IL2, TNFα and IFNy were assessed. TAC T cells readily produce TNFα and IFNy following recognition of its cognate ligand. TAC T cells coexpressing the TNFR1-4-1BB CCR appear to yield fewer cytokine secreting cells.

FIGS. 13A-B: Proliferation and Enrichment of TAC+CCR Engineered T Cells

FIG. 13A illustrates CellTrace™ Violet histogram depicting the dilution peaks of proliferating engineered T cells. FIG. 13B shows quantification of division index and proliferation index of CD4 and CD8 T cells. Data points indicate paired proliferation assays. Division index represents the average number of T cells that a dividing cell became. Proliferation assay represents average number of T cells that an initial cell became. CD8+ and CD4+ T cells engineered to express the BCMA-specific TAC and the TNFR1-4-1BB CCR (TAC+COSTIM) exhibited higher division index values relative to T cells expressing the BCMA-specific TAC alone (TAC). CD4+ T cells engineered to express the BCMA-specific TAC and the TNFR1-4-1BB CCR (TAC+COSTIM) exhibited higher proliferation index values than CD4+ T cells expressing the BCMA-specific TAC alone (TAC). (*p<0.05).

FIG. 14: Anti-CD3 Stimulated Proliferation for TNFRSF screen

PBMCs stimulated with anti-CD3/anti-CD28 beads and grown in IL2 and IL7 were engineered with the TNFR1-4-1BB CCR, TNF-Blocker, and NGFR (transduction control). On day 14 of culture engineered cells were assessed for proliferation following plate bound anti-CD3 stimulation for 5 days. Cells were labelled with CellTrace™ Violet and generation peaks were evaluated with Flow Cytometry. Plate bound anti-CD3 stimulated proliferation in both CD4 and CD8 T cells in all groups. CD4+ and CD8+ T cells engineered with the TNFR1-4-1BB CCR engineered were more proliferative than NGFR or TNF-Blocker engineered cells.

FIG. 15: TNFRSF CCR Engineering Efficiency of CD4+ and CD8+ T cells

PBMCs stimulated with anti-CD3/anti-CD28 beads and grown in IL2 and IL7 were engineered with the TNFRSF CCR constructs listed on the Y-axis. On day 14 of culture the bulk cell populations were assessed for engineering efficiency. Cells were stained for NGFR, the transduction marker included in the gene cassette. Cells were gated on lymphocytes/single cells/CD4+ or CD8+/NGFR+. (N=1-5).

FIG. 16: Mean Fluorescence Intensity of CCRs

PBMCs stimulated with anti-CD3/anti-CD28 beads and grown in IL2 and IL7 were engineered with the TNFRSF CCR screen constructs listed on the Y-axis. On day 14 of culture, engineered cells were assessed for CCR surface expression. Cells were stained for the extracellular portion of the CCR (TNFR1). The CCR MFI was calculated on the population gated lymphocytes/single cells/CD4+ or CD8+/NGFR+. The CCR MFI is reported normalized to the original TNFR1-4-1BB CCR (0G4-1BB) MFI to correct for inter-experimental batch effects. (N=1-5).

FIG. 17: Growth of CCR T cells

PBMCs stimulated with anti-CD3/anti-CD28 beads and grown in IL2 and IL7 were engineered with the TNFRSF CCR screen constructs listed on the Y-axis. Cultures were fed with fresh media and cytokine every two days. On day 14 of culture, live cell counts were recorded for the bulk cell population. Cultures were started with 10⁵ PBMCs. NGFR-transduced control is marked in red. (N=1-5).

FIG. 18: CD4 CCR T Cell Proliferation

PBMCs stimulated with anti-CD3/anti-CD28 beads and grown in IL2 and IL7 were engineered with the TNFRSF CCR screen constructs listed on the Y-axis. On day 14 of culture, engineered cells were assessed for proliferation following stimulation with plate bound anti-CD3 for 5 days. Cells were labelled with CellTrace™ Violet and generation peaks were evaluated by Flow Cytometry. Proliferation was quantified by FCS Express® proliferation modelling and is represented by the Proliferation Index. Control cells engineered with NGFR transduction marker alone are identified (red line). The cell population gated on live cells/lymphocytes/single cells/CD4+/NGFR+. NGFR-transduced control is marked in red.

FIG. 19: CD8 CCR T cell Proliferation Screen

PBMCs stimulated with anti-CD3/anti-CD28 beads and grown in IL2 and IL7 were engineered with the TNFRSF CCR screen constructs listed on the Y-axis. On day 14 of culture, engineered cells were assessed for proliferation following stimulation with plate bound anti-CD3 for 5 days. Cells were labelled with CellTrace™ Violet and generation peaks were evaluated by Flow Cytometry. Proliferation was quantified by FCS Express® proliferation modelling and is represented by the Proliferation Index. Control cells engineered with NGFR transduction marker alone are identified (red line). The cell population gated on live cells/lymphocytes/single cells/CD8+/NGFR+. NGFR-transduced control is marked in red.

FIG. 20: Correlation Heatmap of Clustering Centroids for CCR Screen Constructs

Phenotypic and functional data of TNFRSF CCR engineered T cells were analyzed by Principle Component Analysis (PCA) and K-means clustering. A correlation heatmap of clustering centroids groups CCR with similar attributes together. Different transmembrane and signaling domains are shown as different colors to demonstrate grouping in the dendrogram. Groups identified in the analysis serve to highlight CCRs important for further investigation.

FIGS. 21A-B: Proliferation of CCR T Cells

PBMCs stimulated with anti-CD3/anti-CD28 beads and grown in IL2 and IL7 were engineered with TNFR1-4-1BB, TNFR1-BAFFR, TNF-Blocker, or NGFR (transduction control). On day 14 of culture, engineered cells were assessed for proliferation following stimulation with plate bound anti-CD3 for 5 days. Cells were labelled with CellTrace™ Violet and generation peaks were evaluated with Flow Cytometry. Proliferation was quantified by FCS Express® Proliferation Modelling and is represented by the Proliferation Index. Values shown are normalized within-donor to NGFR (transduction control). In all conditions, plate bound anti-CD3 stimulation resulted in proliferation in both CD4 and CD8 T cell subsets. FIG. 21A shows CD4 T cells engineered with TNFR-BAFFR and TNFR1-4-1BB CCR were more proliferative than NGFR or TNF-Blocker engineered cells over 5 days. FIG. 21B CD8 T cells engineered with TNFR-BAFFR and TNFR1-4-1BB CCR were more proliferative than NGFR or TNF-Blocker engineered cells over 5 days. (*=p<0.05).

FIGS. 22A-B: Intracellular Cytokine Production of CCR Engineered T Cells

T cells engineered with CCR receptors were tested on day 14 of culture for cytokine production following anti-CD3 stimulation. Cells were incubated on anti-CD3 coated plates for 4 hrs in the presence of a Golgi transport inhibitor (GolgiPlug®). Cells were stained and assessed by flow Cytometry for intracellular cytokine production. FIG. 22A shows A high percentage of T cells secrete IFNy upon stimulation. T cells engineered with either 4-1BB/BAFFR CCRs or TNF-Blocker did not alter IFNy production as compared to NGFR transduced control. FIG. 22B shows a high percentage of 531 (NGFR transduced Control) engineered T cells produce TNFα upon anti-CD3 stimulation. Cells engineered with a TNF-Blocker, BAFFR or 4-1BB CCR have reduced amounts of T cells expressing TNFα following stimulation in both CD4+ and CD8+ T cell subsets.

FIGS. 23A-C: TNFR fusion constructs

Representative diagrams of the TNFR-fusion constructs for PCR and ligation into pCCL vectors. FIG. 23A illustrates the full length TNFR1 native receptor was cloned for use as a control receptor in the characterization of TNFR-fusions. FIG. 23B illustrates the CCR receptor was cloned by fusing both the extracellular and transmembrane domain of the TNFR1 receptor to the intracellular signaling domain of 4-1BB, creating a costimulatory TNFR1-4-1BB CCR receptor. FIG. 23C illustrates for use as a dominant negative receptor the native TNFR1 receptor was truncated to remove the cytoplasmic signaling domain and was termed the TNF-Blocker receptor.

FIG. 24: Engineering Efficiency in Fas-Chimera T cells

Human PBMCs activated with anti-CD3/anti-CD28 beads were transduced with either lentiviruses encoding truncated Fas (FasR-TRUNC) or Fas chimeras comprising the extracellular and transmembrane domains of Fas and the cytoplasmic domains of 4-1BB or BAFF-R (Fas-41BB and Fas-BAFFR, respectively) as described in FIG. 2B. As a control, PBMC were transduced with a lentivirus encoding NGFR. Cells were grown in IL2/IL7 with fresh media added every two days. On day 14 of culture, cells were collected and stained for transduction efficiency via flow cytometry. X-axis of plot depicts T cells engineered to express the named receptor. (n=3, independent experiments)

FIGS. 25A-B: Surface Expression of Fas Chimera Engineered T cells

Human PBMCs activated with anti-CD3/anti-CD28 beads were transduced with either lentiviruses encoding truncated Fas (FasR-TRUNC) or Fas chimeras comprising the extracellular and transmembrane domains of Fas and the cytoplasmic domains of 4-1BB or BAFF-R (Fas-41BB and Fas-BAFFR, respectively) as described in FIG. 2B. As a control, PBMC were transduced with a lentivirus encoding NGFR. Cells were grown in IL2/IL7 with fresh media added every two days. On day 14 of culture, cells were collected and stained for Fas via flow cytometry. FIG. 25A. Dot plots of Fas and NGFR expression in Fas-chimera engineered T cells, depicted as % NGFR-positive cells that display Fas above baseline expression. FIG. 25B. Bar plot of Fas expression (MFI) in engineered T cells. Fas expression above NGFR is an indirect measure of the modified Fas receptors. X-axis depicts T cells engineered to express the named receptor. ((n=3), independent experiments)

FIG. 26: Growth of Fas-chimera T cells during initial expansion

Human PBMCs activated with anti-CD3/anti-CD28 beads were transduced with either lentiviruses encoding truncated Fas (FasR-TRUNC) or Fas chimeras comprising the extracellular and transmembrane domains of Fas and the cytoplasmic domains of 4-1BB or BAFF-R (Fas-41BB and Fas-BAFFR, respectively) as described in FIG. 2B. As a control, PBMC were transduced with a lentivirus encoding NGFR. Cells were grown in IL2/IL7 supplemented with fresh media every two days. On day 14 of culture cell expansion was measured as total live cell count. The x-axis depicts T cells engineered to express the named receptor. (n=3, independent experiments)

FIG. 27: Proliferation of Fas engineered T cells

Human PBMCs activated with anti-CD3/anti-CD28 beads were engineered to express the modified Fas receptors (Fas-TRUNC, Fas-4-1BB, Fas-BAFFR) or no receptor (NGFR). The engineered T cells were labeled with Cell Trace Violet, stimulated with anti-CD3 and cultured for 4 days. Proliferation was quantified by FCS Express® Proliferation Modelling and is represented by the Proliferation Index normalized to non-engineered. The x-axis depicts stimulated T cells engineered to express CD8 (FIG. 27A) or CD4 (FIG. 27B). (n=3, independent experiments).

FIG. 28: Viability of Fas-chimera T cells in the presence of FasL

Human PBMCs activated with anti-CD3/anti-CD28 beads were engineered to express the modified Fas receptors (Fas-TRUNC, Fas-4-1BB, Fas-BAFFR) or no receptor (NGFR). The engineered T cells were cultured in the presence of increasing concentrations of FasL. Viability was measured after 48 hrs by AlamarBlue. (n=3 replicates, Figure representative of 3 independent experiments).

FIG. 29: AlamarBlue assay of Proliferating Fas engineered T cells

Human PBMCs activated with anti-CD3/anti-CD28 beads were engineered to express the modified Fas receptors (Fas-TRUNC, Fas-4-1BB, Fas-BAFFR) or no receptor (NGFR). The engineered T cells were labeled with Cell Trace Violet, stimulated with anti-CD3 and cultured for 4 days in the presence or absence of increasing concentrations of FasL (x-axis). Proliferation was measured by AlamarBlue. Legend: T cells engineered with the named receptor. (n=3 replicates, representative of 3 independent experiments).

### DETAILED DESCRIPTION

### Definitions

The term “a cell” as used herein includes a single cell as well as a plurality of cells.

The term “T cell” as used herein refers to a type of lymphocyte that plays a central role in cell-mediated immunity. T cells, also referred to as T lymphocytes, can be distinguished from other lymphocytes, such as B cells and natural killer cells, by the presence of a T-cell receptor (TCR) on the cell surface. There are several subsets of T cells with distinct functions, including but not limited to, T helper cells, cytotoxic T cells, memory T cells, regulatory T cells and natural killer T cells. In some embodiments, the T cell is an engineered T cell.

The term “engineered TCR” or “engineered T-cell receptor” means any TCR that has been modified from its naturally-occurring form. An engineered TCR may have modifications to the alpha and/or beta chains, or the gamma and/or delta chains (including replacement of any of the aforementioned chains) that enable the TCR to recognize a specific antigen (for example, a neoantigen). The engineered TCR may have modifications to any CD3 subunit (for example, CD3c, as in the case of TRuC receptors), including the addition of an antigen recognition domain (e.g., an antibody, an scFv, a DARPin). The engineered TCR may have an antigen recognition domain (e.g., an antibody, an scFv, a DARPin) joined to a transmembrane domain of the alpha and/or beta chains, or the gamma and/or delta chains.

The term “polynucleotide” and/or “nucleic acid sequence” and/or “nucleic acid” as used herein refers to a sequence of nucleoside or nucleotide monomers consisting of bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acids of the present disclosure may be isolated from biological organisms, formed by laboratory methods of genetic recombination or obtained by chemical synthesis or other known protocols for creating nucleic acids.

The term “isolated polynucleotide” or “isolated nucleic acid sequence” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded, and represents the sense or antisense strand. Further, the term “nucleic acid” includes the complementary nucleic acid sequences.

The term “recombinant nucleic acid” or “engineered nucleic acid” as used herein refers to a nucleic acid or polynucleotide that is not found in a biological organism. For example, recombinant nucleic acids may be formed by laboratory methods of genetic recombination (such as molecular cloning) to create sequences that would not otherwise be found in nature. Recombinant nucleic acids may also be created by chemical synthesis or other known protocols for creating nucleic acids. Unless otherwise indicated, the definitions and embodiments described in this and other sections are intended to be applicable to all embodiments and aspects of the present application herein described for which they are suitable as would be understood by a person skilled in the art.

The term “polypeptide” or “protein” as used herein describes a chain of amino acids that correspond to those encoded by a nucleic acid. A polypeptide or protein of this disclosure can be a peptide, which usually describes a chain of amino acids of from two to about 30 amino acids. The term protein as used herein also describes a chain of amino acids having more than 30 amino acids and can be a fragment or domain of a protein or a full length protein. Furthermore, as used herein, the term protein can refer to a linear chain of amino acids or it can refer to a chain of amino acids that has been processed and folded into a functional protein. It is understood, however, that 30 is an arbitrary number with regard to distinguishing peptides and proteins and the terms can be used interchangeably for a chain of amino acids. The proteins of the present disclosure can be obtained by isolation and purification of the proteins from cells where they are produced naturally, by enzymatic (e.g., proteolytic) cleavage, and/or recombinantly by expression of nucleic acid encoding the proteins or fragments of this disclosure. The proteins and/or fragments of this disclosure can also be obtained by chemical synthesis or other known protocols for producing proteins and fragments.

The term “isolated polypeptide” refers to a polypeptide substantially free of cellular material or culture medium when produced by recombinant DNA techniques, or chemical precursors or other chemicals when chemically synthesized.

The term “vector” as used herein refers to a polynucleotide that can be used to deliver a nucleic acid to the inside of a cell. In one embodiment, a vector is an expression vector comprising expression control sequences (for example, a promoter) operatively linked to a nucleic acid to be expressed in a cell. Vectors known in the art include, but are not limited to, plasmids, phages, cosmids and viruses.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

As used herein, the terms “treatment,” “treating,” and the like, in some embodiments, refer to administering an agent, or carrying out a procedure, for the purposes of obtaining an effect. The effect may be prophylactic in terms of completely or partially preventing a disease or symptom thereof and/or may be therapeutic in terms of affecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms; or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent, delay, alleviate, arrest or inhibit development of the symptoms or conditions associated with diseases (e.g. cancer). The term “therapeutic effect” refers to the reduction, elimination, or prevention of the disease, symptoms of the disease, or side effects of the disease in the subject.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “and/or” as used herein means that the listed items are present, or used, individually or in combination. In effect, this term means that “at least one of” or “one or more” of the listed items is used or present.

Chimeric Costimulatory Receptors (CCRs)

Disclosed herein, in certain embodiments, are Chimeric Costimulatory Receptor (CCR) nucleic acids, comprising (a) a first polynucleotide encoding an extracellular domain of a member of the Tumor Necrosis Factor Receptor Superfamily (TNFRSF); (b) a second polynucleotide encoding a transmembrane domain polypeptide; and (c) a third polynucleotide encoding a cytosolic costimulatory signaling domain polypeptide (e.g., from a Tumor Necrosis Factor Receptor Superfamily (TNFRSF) member). In some embodiments, the first polynucleotide and the third polynucleotide are derived from different members of the TNFRSF.

In some embodiments, the first polynucleotide encodes an extracellular domain of a member of the TNFRSF. In some embodiments, the first polynucleotide and the third polynucleotide are derived from different members of the TNFRSF. In some embodiments, the first polynucleotide encodes an extracellular domain of TNFRSF member having a death domain. In some embodiments, the first polynucleotide encodes an extracellular domain of TNFR1, TNFR2, Fas, DR4, DR5, DR3, DR6, EDAR, XEDAR, TROY or NGFR. In some embodiments, the first polynucleotide encodes an extracellular domain of TNFR1. In some embodiments, the first polynucleotide encodes an extracellular domain of TNFR2. In some embodiments, the first polynucleotide encodes an extracellular domain of Fas. In some embodiments, the first polynucleotide encodes an extracellular domain of DR4. In some embodiments, the first polynucleotide encodes an extracellular domain of DR5. In some embodiments, the first polynucleotide encodes an extracellular domain of DR3. In some embodiments, the first polynucleotide encodes an extracellular domain of DR6. In some embodiments, the first polynucleotide encodes an extracellular domain of EDAR. In some embodiments, the first polynucleotide encodes an extracellular domain of XEDAR. In some embodiments, the first polynucleotide encodes an extracellular domain of TROY. In some embodiments, the first polynucleotide encodes an extracellular domain of NGFR.

The tumor necrosis factor receptor superfamily (TNFRSF) is a protein superfamily of cytokine receptors characterized by the ability to bind tumor necrosis factors (TNFs) via an extracellular cysteine-rich domain. There are 27 members of the TNFR Superfamily, including TNFR1, TNFR2, Fas, DR4, DR5, DR3, DR6, EDAR, XEDAR, TROY or NGFR.

Tumor necrosis factor receptor 1 (TNFR1), also known as tumor necrosis factor receptor superfamily member 1A (TNFRSF1A) and CD120a, is a membrane-bound receptor that binds tumor necrosis factor-alpha (TNFα). TNFR1 activates the transcription factor NF-κB, mediates apoptosis, and functions as a regulator of inflammation.

Tumor necrosis factor receptor 2 (TNFR2), also known as tumor necrosis factor receptor superfamily member 1B (TNFRSF1B) and CD120b, is a membrane-bound receptor that binds tumor necrosis factor-alpha (TNFα).

The Fas receptor, also known as Fas, FasR, apoptosis antigen 1 (APO-1 or APT), cluster of differentiation 95 (CD95) or tumor necrosis factor receptor superfamily member 6 (TNFRSF6), is a protein that in humans is encoded by the FAS gene. Multiple splice variants of Fas have been identified, which are translated into seven isoforms of the protein. Apoptosis-inducing Fas receptor is dubbed isoform 1 and is a type 1 transmembrane protein. Many of the other isoforms are rare haplotypes that are usually associated with a state of disease. Any suitable isoform of Fas is contemplated for use with the embodiments disclosed herein.

Death domain 4 (DR4), also known as TRAIL receptor 1 (TRAILR1) and tumor necrosis factor receptor superfamily member 10A (TNFRSF10A), is a cell surface receptor of the TNF-receptor superfamily that binds TRAIL and mediates apoptosis.

Death domain 5 (DR5), also known as TRAIL receptor 2 (TRAILR2) and tumor necrosis factor receptor superfamily member 10B (TNFRSF10B), is a cell surface receptor of the TNF-receptor superfamily that binds TRAIL and mediates apoptosis.

Death domain 3 (DR3), also known as tumor necrosis factor receptor superfamily member 25 (TNFRSF25), is a cell surface receptor of the tumor necrosis factor receptor superfamily which mediates apoptotic signalling and differentiation. Its only known TNFSF ligand is TNF-like protein 1A (TL1A).

Death domain 6 (DR6), also known as tumor necrosis factor receptor superfamily member 21 (TNFRSF21), is a cell surface receptor of the tumor necrosis factor receptor superfamily which activates the JNK and NF-κB pathways.

Ectodermal dysplasia receptor (EDAR) is a member of the TNF-receptor superfamily. It plays a key role in the process of ectodermal differentiation.

Ectodysplasin A2 receptor (XEDAR; Tumor necrosis factor receptor superfamily member 27) is a protein that in humans is encoded by the EDA2R gene. EDA-A1 and EDA-A2 are two isoforms of ectodysplasin that are encoded by the anhidrotic ectodermal dysplasia (EDA) gene.

TROY (Tumor necrosis factor receptor superfamily member 19, TNFRSF19) is a member of the TNF-receptor superfamily. This receptor is highly expressed during embryonic development. It has been shown to interact with TNF receptor associated factor (TRAF) family members, and to activate c-Jun N-terminal kinases (JNK) signaling pathway when overexpressed in cells. This receptor is capable of inducing apoptosis by a caspase-independent mechanism, and it is thought to play an essential role in embryonic development.

NGFR (low-affinity nerve growth factor receptor; nerve growth factor receptor; TNFR superfamily member 16); LNGFR; p75 neurotrophin receptor) is a member of the tumor necrosis factor receptor (TNF receptor) superfamily. It is one of the two receptor types for the neurotrophins, a family of protein growth factors that stimulate neuronal cells to survive and differentiate.

In some embodiments, the first polynucleotide encodes an oligopeptide having a sequence according to SEQ ID NO: 1. In some embodiments, the polynucleotide encodes an oligopeptide having a sequence having at least 80%, 85%, 90%, 95%, or 99% identity to a sequence according to SEQ ID NO: 1. In some embodiments, the first polynucleotide encodes an oligopeptide having a sequence according to SEQ ID NO: 2. In some embodiments, the polynucleotide encodes an oligopeptide having a sequence having at least 80%, 85%, 90%, 95%, or 99% identity to a sequence according to SEQ ID NO: 2.

| SEQ ID | Domain Name | AA Sequence |
|---|---|---|
| SEQ ID | TNFR1 | GLSTVPDLLLPLVLLELLVGIYP |
| NO: 1 | extracellular | SGVIGLVPHLGDREKRDSVCPQG |
| | domain | KYIHPQNNSICCTKCHKGTYLYN |
| | | DCPGPGQDTDCRECESGSFTASE |
| | | NHLRHCLSCSKCRKEMGQVEISS |
| | | CTVDRDTVCGCRKNQYRHYWSEN |
| | | LFQCFNCSLCLNGTVHLSCQEKQ |
| | | NTVCTCHAGFFLRENECVSCSNC |
| | | KKSLECTKLCLPQIENVKGTEDS |
| | | GTT |
| SEQ ID | FAS receptor | MLGIWTLLPLVLTSVARLSSKSV |
| NO: 2 | extracellular | NAQVTDINSKGLELRKTVTTVET |
| | domain | QNLEGLHHDGQFCHKPCPPGERK |
| | | ARDCTVNGDEPDCVPCQEGKEYT |
| | | DKAHFSSKCRRCRLCDEGHGLEV |
| | | EINCTRTQNTKCRCKPNFFCNST |
| | | VCEHCDPCTKCEHGIIKECTLTS |
| | | NTKCKEEGSRSN |

In some embodiments, the third polynucleotide encodes a cytosolic costimulatory signaling domain polypeptide from CD28, CD28T, OX-40, 4-1BB/CD137, CD2, CD7, CD27, CD30, CD40, Programmed Death-1 (PD-1), inducible T cell costimulator (ICOS), lymphocyte function-associated antigen-1 (LFA-1, CD1-CD18), CD247, CD276 (B7-H3), LIGHT, (TNFSF14), NKG2C, Ig alpha (CD79a), DAP-10, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD 103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, or CD19a.

In some embodiments, the third polynucleotide encodes a cytosolic costimulatory signaling domain from 4-1BB, BAFFR, OX40, CD27, CD40, GITR, HVEM, OX40, RELT, TACI, TROY, or TWEAK. In some embodiments, the third polynucleotide encodes a cytosolic costimulatory signaling domain from BAFFR. In some embodiments, the third polynucleotide encodes a cytosolic costimulatory signaling domain from 4-1BB. In some embodiments, the third polynucleotide encodes a cytosolic costimulatory signaling domain from CD27. In some embodiments, the third polynucleotide encodes a cytosolic costimulatory signaling domain from HVEM. In some embodiments, the third polynucleotide encodes a cytosolic costimulatory signaling domain from OX40. In some embodiments, the third polynucleotide encodes a cytosolic costimulatory signaling domain from GITR. In some embodiments, the third polynucleotide encodes a cytosolic costimulatory signaling domain from TACI.

In some embodiments, the third polynucleotide encodes an oligopeptide having a sequence according to SEQ ID NO: 3. In some embodiments, the polynucleotide encodes an oligopeptide having a sequence having at least 80%, 85%, 90%, 95%, or 99% identity to a sequence according to SEQ ID NO: 3. In some embodiments, the third polynucleotide encodes an oligopeptide having a sequence according to SEQ ID NO: 4. In some embodiments, the polynucleotide encodes an oligopeptide having a sequence having at least 80%, 85%, 90%, 95%, or 99% identity to a sequence according to SEQ ID NO: 4.

| SEQ ID | Domain Name | AA Sequence |
|---|---|---|
| SEQ ID | 4-IBB | KRGRKKLLYIFKQPFMRPVQTTQEED |
| NO: 3 | co-stimulatory | GCSCRFPEEEEGGCEL |
| | signaling | |
| | domain | |
| SEQ ID | BAFFR | SWRRRQRRLRGASSAEAPDGDKDAPE |
| NO: 4 | co-stimulatory | PLDKVIILSPGISDATAPAWPPPGED |
| | signaling | PGTTPPGHSVPVPATELGSTELVTTK |
| | domain | TAGPEQQ |

In some embodiments, the second polynucleotide encodes a transmembrane domain from a member of the TNFR Superfamily. In some embodiments, the second polynucleotide encodes a transmembrane domain from 4-1BB, BAFFR, OX40, CD27, CD40, GITR, HVEM, OX40, RELT, TACI, TROY, or TWEAK. In some embodiments, the transmembrane domain is derived from a different molecule than the cytosolic costimulatory signaling domain. In some embodiments, the transmembrane domain is derived from the same molecule as the cytosolic costimulatory signaling domain. In some embodiments, the transmembrane domain and the cytosolic costimulatory signaling domain are derived from the same costimulatory molecule selected from: 4-1BB, BAFFR, OX40, CD27, CD40, GITR, HVEM, OX40, RELT, TACI, TROY, and TWEAK.

In some embodiments, the second polynucleotide encodes an oligopeptide having a sequence according to SEQ ID NO: 5. In some embodiments, the polynucleotide encodes an oligopeptide having a sequence having at least 80%, 85%, 90%, 95%, or 99% identity to a sequence according to SEQ ID NO: 5. In some embodiments, the second polynucleotide encodes an oligopeptide having a sequence according to SEQ ID NO: 6. In some embodiments, the polynucleotide encodes an oligopeptide having a sequence having at least 80%, 85%, 90%, 95%, or 99% identity to a sequence according to SEQ ID NO: 6.

| SEQ ID | Domain Name | AA Sequence |
|---|---|---|
| SEQ ID | TNFR1 TM | VLLPLVIFFGLCLLSLL |
| NO: 5 | domain | FIGL |
| SEQ ID | Fas Receptor | LGWLCLLLLPIPLIVWV |
| NO: 6 | TM domain | |

Single Molecule

In some embodiments, the CCR is a single molecule. In some embodiments, the first polynucleotide and second polynucleotide are joined directly or indirectly (e.g., via a linker) to the third polynucleotide. In some embodiments, the first polynucleotide and third polynucleotide are joined directly or indirectly (e.g., via a linker) to the second polynucleotide. Any suitable linker is contemplated for use with the molecules disclosed herein. In some embodiments, the linker is a small molecule. In some embodiments, the linker is a peptide linker.

In some embodiments, the CCR has an amino acid sequence according to SEQ ID NO: 7. In some embodiments, the polynucleotide encodes an oligopeptide having a sequence having at least 80%, 85%, 90%, 95%, or 99% identity to a sequence according to SEQ ID NO: 7. In some embodiments, the CCR has an amino acid sequence according to SEQ ID NO: 8. In some embodiments, the polynucleotide encodes an oligopeptide having a sequence having at least 80%, 85%, 90%, 95%, or 99% identity to a sequence according to SEQ ID NO: 8. In some embodiments, the CCR has an amino acid sequence according to SEQ ID NO: 9. In some embodiments, the polynucleotide encodes an oligopeptide having a sequence having at least 80%, 85%, 90%, 95%, or 99% identity to a sequence according to SEQ ID NO: 9. In some embodiments, the CCR has an amino acid sequence according to SEQ ID NO: 10. In some embodiments, the polynucleotide encodes an oligopeptide having a sequence having at least 80%, 85%, 90%, 95%, or 99% identity to a sequence according to SEQ ID NO: 10.

| SEQ ID | | AA Sequence |
|---|---|---|
| SEQ ID | TNFR1 | GLSTVPDLLLPLVLLELLVGIYPSG |
| NO: 7 | extracellular | VIGLVPHLGDREKRDSVCPQGKYIH |
| | domain/TNFR1 TM | PQNNSICCTKCHKGTYLYNDCPGPG |
| | domain/4-lBB | QDTDCRECESGSFTASENHLRHCLS |
| | costimulatory | CSKCRKEMGQVEISSCTVDRDTVCG |
| | intracellular | CRKNQYRHYWSENLFQCFNCSLCLN |
| | signaling domain | GTVHLSCQEKQNTVCTCHAGFFLRE |
| | | NECVSCSNCKKSLECTKLCLPQIEN |
| | | VKGTEDSGTTVLLPLVIFFGLCLLS |
| | | LLFIGLKRGRKKLLYIFKQPFMRPV |
| | | QTTQEEDGCSCRFPEEEEGGCEL |
| SEQ ID | TNFR1 | GLSTVPDLLLPLVLLELLVGIYPSG |
| NO: 8 | extracellular | VIGLVPHLGDREKRDSVCPQGKYIH |
| | domain/TNFR1 TM | PQNNSICCTKCHKGTYLYNDCPGPG |
| | domain/BAFF-R | QDTDCRECESGSFTASENHLRHCLS |
| | costimulatory | CSKCRKEMGQVEISSCTVDRDTVCG |
| | intracellular | CRKNQYRHYWSENLFQCFNCSLCLN |
| | signaling domain | GTVHLSCQEKQNTVCTCHAGFFLRE |
| | | NECVSCSNCKKSLECTKLCLPQIEN |
| | | VKGTEDSGTTVLLPLVIFFGLCLLS |
| | | LLFIGLSWRRRQRRLRGASSAEAPD |
| | | GDKDAPEPLDKVIILSPGISDATAP |
| | | AWPPPGEDPGTTPPGHSVPVPATEL |
| | | GSTELVTTKTAGPEQQ |
| SEQ ID | Fas receptor | MLGIWTLLPLVLTSVARLSSKSVNA |
| NO: 9 | extracellular | QVTDINSKGLELRKTVTTVETQNLE |
| | domain/Fas | GLHHDGQFCHKPCPPGERKARDCTV |
| | receptor | NGDEPDCVPCQEGKEYTDKAHFSSK |
| | TM domain/4-1BB | CRRCRLCDEGHGLEVEINCTRTQNT |
| | costimulatory | KCRCKPNFFCNSTVCEHCDPCTKCE |
| | intracellular | HGIIKECTLTSNTKCKEEGSRSNLG |
| | signaling domain | WLCLLLLPIPLIVWVKRGRKKLLYI |
| | | FKQPFMRPVQTTQEEDGCSCRFPEE |
| | | EEGGCEL |
| SEQ ID | Fas receptor | MLGIWTLLPLVLTSVARLSSKSVNA |
| NO: 10 | extracellular | QVTDINSKGLELRKTVTTVETQNLE |
| | domain/Fas | GLHHDGQFCHKPCPPGERKARDCTV |
| | receptor | NGDEPDCVPCQEGKEYTDKAHFSSK |
| | TM domain/BAFF-R | CRRCRLCDEGHGLEVEINCTRTQNT |
| | costimulatory | KCRCKPNFFCNSTVCEHCDPCTKCE |
| | intracellular | HGIIKECTLTSNTKCKEEGSRSNLG |
| | signaling domain | WLCLLLLPIPLIVWVSWRRRQRRLR |
| | | GASSAEAPDGDKDAPEPLDKVIILS |
| | | PGISDATAPAWPPPGEDPGTTPPGH |
| | | SVPVPATELGSTELVTTKTAGPEQQ |

Immune Cells

Disclosed herein, in certain embodiments, are immune cells, comprising a Chimeric Costimulatory Receptor (CCR) nucleic acid, comprising (a) a first polynucleotide encoding an extracellular domain of a member of the Tumor Necrosis Factor Receptor Superfamily; (b) a second polynucleotide encoding a transmembrane domain polypeptide; and (c) a third polynucleotide encoding a cytosolic costimulatory signaling domain polypeptide (e.g., from the Tumor Necrosis Factor Receptor Superfamily (TNFRSF) member). In some embodiments, the first polynucleotide and the third polynucleotide are derived from different members of the TNFRSF. In some embodiments, the first polynucleotide encodes an extracellular domain of TNFRSF member having a death domain. In some embodiments, the first polynucleotide encodes an extracellular domain of TNFR1, TNFR2, Fas, DR4, DR5, DR3, DR6, EDAR, XEDAR, TROY or NGFR. In some embodiments, the third polynucleotide encodes a cytosolic costimulatory signaling domain polypeptide from 4-1BB, BAFFR, OX40, CD27, CD40, GITR, HVEM, OX40, RELT, TACI, TROY, or TWEAK. In some embodiments, the immune cell is a T cell (e.g., cytotoxic T cell, helper T cell, regulatory T cell, gamma-delta T cell). In some embodiments, the T cell is an engineered T cell. In some embodiments, the immune cell is a natural killer cell (NK cell). In some embodiments, the immune cell is a macrophage. In some embodiments, the immune cell is a tumor-infiltrating lymphocyte (TIL). In some embodiments, the immune cell is a monocyte. In some embodiments, the immune cell is a B cell.

T-Cells

Disclosed herein, in certain embodiments, are T cells, comprising a Chimeric Costimulatory Receptor (CCR) nucleic acid comprising (a) a first polynucleotide encoding an extracellular domain of a member of the Tumor Necrosis Factor Receptor Superfamily; (b) a second polynucleotide encoding a transmembrane domain polypeptide; and (c) a third polynucleotide encoding a cytosolic costimulatory signaling domain polypeptide (e.g., from a Tumor Necrosis Factor Receptor Superfamily (TNFRSF) member). In some embodiments, the first polynucleotide and the third polynucleotide are derived from different members of the TNFRSF. In some embodiments, the first polynucleotide encodes an extracellular domain of TNFRSF member having a death domain. In some embodiments, the first polynucleotide encodes an extracellular domain of TNFR1, TNFR2, Fas, DR4, DR5, DR3, DR6, EDAR, XEDAR, TROY or NGFR. In some embodiments, the third polynucleotide encodes a cytosolic costimulatory signaling domain polypeptide from 4-1BB, BAFFR, OX40, CD27, CD40, GITR, HVEM, OX40, RELT, TACI, TROY, or TWEAK.

In some embodiments, the T cell is a cytotoxic T cell, helper T cell, regulatory T cell, or a gamma-delta T cell. In some embodiments, the T cell comprises a second nucleic acid encoding an engineered T cell receptor (TCR) or a synthetic antigen receptor polypeptide that can recognize a target-specific ligand. In some embodiments, the synthetic antigen receptor polynucleotide is a Chimeric Antigen Receptor (CAR). In some embodiments, the synthetic antigen receptor polynucleotide encodes a T cell Antigen Coupler (TAC).

In some embodiments, the CAR comprises an antigen binding domain, a transmembrane domain, and an intracellular signaling domain (e.g., an intracellular signaling domain comprising a costimulatory domain and/or a primary signaling domain). In some embodiments, the transmembrane domain is a transmembrane domain selected from the group consisting of the alpha or beta of the T-cell receptor, CD28, CD3 epsilon, CD3 zeta, CD45, CD4, CD5, CD8, CD9, CD16, CD22, CD33, CD37, CD64, CD80, CD86, CD134, CD 137 and CD 154. In some embodiments, the intracellular signaling domain is a signaling domain derived from CD28, CD28T, OX-40, 4-1BB/CD137, CD2, CD7, CD27, CD30, CD40, Programmed Death-1 (PD-1), inducible T cell costimulator (ICOS), lymphocyte function-associated antigen-1 (LFA-1, CD1-CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD 103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, or CD19a.

In some embodiments, the TAC comprises an antigen binding domain, a domain that binds a protein associated with the TCR complex, and a T cell co-receptor domain comprising a cytosolic domain and a transmembrane domain. In some embodiments, the protein associated with the TCR complex is CD3. In some embodiments, the TAC does not comprise a costimulatory domain and/or an activation domain. In some embodiments, the cytosolic domain is a CD4 cytosolic domain and the transmembrane domain is a CD4 transmembrane domain. In some embodiments, the TAC comprises an antigen binding domain, a CD3 binding domain, and a CD4 cytosolic domain and CD4 transmembrane domain. In some embodiments, the CD3 antigen binding domain is derived from UCHT1.

In some embodiments, the engineered TCR is a T cell receptor (TCR) fusion protein (TFP). In some embodiments, the TFP comprises at least one engineered CD3 chain, the engineered CD3 chain comprising (a) at least a portion of an extracellular domain, (b) an intracellular domain comprising a stimulatory domain from an intracellular signaling domain; and (c) an antigen binding domain (e.g., a scFv). In some embodiments, the extracellular domain and the intracellular domain are derived from CD3α, CD3β, CD3γ, CD3δ, or CD3ε. In some embodiments, the engineered CD3 chain further comprises a transmembrane domain. In some embodiments, the extracellular domain, the transmembrane domain, and the intracellular domains are derived from CD3c. In some embodiments, the engineered CD3 chain replaces at least one naturally occurring CD3c chain of a TCR.

In some embodiments, the engineered TCR is a chimeric antibody-T cell receptor (TCR) construct (caTCR). In some embodiments, the caTCR comprises an antigen-binding module that specifically binds to a target antigen and a T cell receptor module (TCRM) capable of recruiting at least one TCR-associated signaling molecule. In some embodiments, the TCRM comprises a first TCR domain (TCRD) comprising a first TCR transmembrane domain (TCR-TM) and a second TCRD comprising a second TCR-TM, wherein the TCR-TM facilitates recruitment of at least one TCR-associated signaling molecule. In some embodiments, the first TCR-TM is derived from one of the transmembrane domains of a T cell receptor (such as an αβ TCR, or a γδ TCR) and the second TCR-TM is derived from the other transmembrane domain of the T cell receptor. In some embodiments, the TCR is an αβ TCR and the first and second TCR-TMs are derived from TCR α and β subunit transmembrane domains. In some embodiments, the TCR is a γδ TCR and the first and second TCR-TMs are derived from TCR γ and δ subunit transmembrane domains. In some embodiments, TCRD and TCR-TM are fused to an F(ab) derived from an antibody. In some embodiments, TCRD and TCR-TM are fused to a single-chain antibody (scFv) derived from the F(v) portion of an antibody.

In some embodiments, the antigen binding domain binds an antigen on a cancerous cell. In some embodiments, the antigen is differentially expressed on a cancerous cell. In some embodiments, the antigen is upregulated on a cancerous cell. In some embodiments, the antigen is a surface antigen and is not presented by an MHC. In some embodiments, the antigen is an MHC/peptide complex.

In some embodiments, the antigen is HER2 (erbB-2), B-cell maturation antigen (BCMA), CD19, alphafetoprotein (AFP), carcinoembryonic antigen (CEA), CA-125, MUC-1, epithelial tumor antigen (ETA), tyrosinase, melanoma-associated antigen (MAGE), prostate-specific antigen (PSA), glioma-associated antigen, β-human chorionic gonadotropin, thyroglobulin, RAGE-1, MN-CA IX, human telomerase reverse transcriptase, RU1, RU2 (AS), intestinal carboxyl esterase, mut hsp70-2, M-CSF, prostase, PAP, NY-ESO-1, LAGE-la, p53, prostein, PSMA, survivin and telomerase, prostate-carcinoma tumor antigen-1 (PCTA-1), ELF2M, neutrophil elastase, CD22, insulin growth factor (IGF)-I, IGF-II, IGF-I receptor and mesothelin. In some embodiments, the antigen is HER2, BCMA, or CD19. In some embodiments, the antigen is HER2. In some embodiments, the antigen is BCMA. In some embodiments, the antigen is CD19.

Methods of Use

Disclosed herein, in certain embodiments, are methods of treating a cancer in an individual in need thereof, comprising administering to the individual an immune cell comprising Chimeric Costimulatory Receptor (CCR) nucleic acid, comprising (a) a first polynucleotide encoding an extracellular domain of a member of the Tumor Necrosis Factor Receptor Superfamily; (b) a second polynucleotide encoding a transmembrane domain polypeptide; and (c) a third polynucleotide encoding a cytosolic costimulatory signaling domain polypeptide (e.g., from a Tumor Necrosis Factor Receptor Superfamily (TNFRSF) member). In some embodiments, the first polynucleotide and the third polynucleotide are derived from different members of the TNFRSF. In some embodiments, the first polynucleotide encodes an extracellular domain of TNFRSF member having a death domain. In some embodiments, the first polynucleotide encodes an extracellular domain of TNFR1, TNFR2, Fas, DR4, DR5, DR3, DR6, EDAR, XEDAR, TROY or NGFR. In some embodiments, the third polynucleotide encodes a cytosolic costimulatory signaling domain polypeptide from 4-1BB, BAFFR, OX40, CD27, CD40, GITR, HVEM, OX40, RELT, TACI, TROY, or TWEAK. In some embodiments, the immune cell is a T cell (e.g., cytotoxic T cell, helper T cell, regulatory T cell, gamma-delta T cell). In some embodiments, the T cell is an engineered T cell. In some embodiments, the immune cell is a natural killer cell (NK cell). In some embodiments, the immune cell is a macrophage. In some embodiments, the immune cell is a tumor-infiltrating lymphocyte (TIL). In some embodiments, the immune cell is a monocyte. In some embodiments, the immune cell is a B cell.

In some embodiments, the cancer is a solid cancer or liquid cancer. In some embodiments, the cancer is a carcinoma, a blastomas, a melanoma, a sarcoma, a hematological cancers, or a lymphoid malignancy.

In some embodiments, the cancer is a leukemia or lymphoma. In some embodiments, the cancer is mixed lineage leukemia (MLL), chronic lymphocytic leukemia (CLL), acute lymphoblastic leukemia (ALL), large B-cell lymphoma, diffuse large B-cell lymphoma, primary mediastinal B cell lymphoma, high grade B-cell lymphoma, or large B cell lymphoma arising from follicular lymphoma.

In some embodiments, the cancer is a lung cancer, a breast cancer, a colon cancer, multiple myeloma, glioblastoma, gastric cancer, ovarian cancer, stomach cancer, colorectal cancer, urothelial cancer, endometrial cancer, or a melanoma. In some embodiments, the cancer is a lung cancer. In some embodiments, the cancer is a breast cancer. In some embodiments, the cancer is a colon cancer. In some embodiments, the cancer is multiple myeloma. In some embodiments, the cancer is a glioblastoma. In some embodiments, the cancer is a gastric cancer. In some embodiments, the cancer is an ovarian cancer. In some embodiments, the cancer is a stomach cancer. In some embodiments, the cancer is a colorectal cancer. In some embodiments, the cancer is urothelial cancer. In some embodiments, the cancer is an endometrial cancer. In some embodiments, the cancer is a melanoma.

Pharmaceutical Compositions

Disclosed herein, in certain embodiments, are pharmaceutical compositions comprising (a) an immune cell comprising (i) a first polynucleotide encoding an extracellular domain of a member of the Tumor Necrosis Factor Receptor Superfamily; (ii) a second polynucleotide encoding a transmembrane domain polypeptide; and (iii) a third polynucleotide encoding a cytosolic costimulatory signaling domain polypeptide (e.g., from a Tumor Necrosis Factor Receptor Superfamily (TNFRSF) member); and (b) a pharmaceutically acceptable carrier. In some embodiments, the first polynucleotide and the third polynucleotide are derived from different members of the TNFRSF. In some embodiments, the first polynucleotide encodes an extracellular domain of TNFRSF member having a death domain. In some embodiments, the first polynucleotide encodes an extracellular domain of TNFR1, TNFR2, Fas, DR4, DR5, DR3, DR6, EDAR, XEDAR, TROY or NGFR. In some embodiments, the first polynucleotide encodes an extracellular domain of TNFR1. In some embodiments, the first polynucleotide encodes an extracellular domain of TNFR2. In some embodiments, the first polynucleotide encodes an extracellular domain of Fas. In some embodiments, the third polynucleotide encodes a cytosolic costimulatory signaling domain from 4-1BB, BAFFR, OX40, CD27, CD40, GITR, HVEM, OX40, RELT, TACI, TROY, or TWEAK. In some embodiments, the third polynucleotide encodes a cytosolic costimulatory signaling domain from BAFFR. In some embodiments, the third polynucleotide encodes a cytosolic costimulatory signaling domain from 4-1BB. In some embodiments, the transmembrane domain and cytosolic costimulatory signaling domain are from the same costimulatory signaling protein. In some embodiments, the transmembrane and costimulatory signaling domains are derived from 4-1BB, BAFFR, OX40, CD27, CD40, GITR, HVEM, OX40, RELT, TACI, TROY, or TWEAK. In some embodiments, the first polynucleotide and second polynucleotide are joined directly or indirectly (e.g., via a linker) to the third polynucleotide. In some embodiments, the first polynucleotide and third polynucleotide are joined directly or indirectly (e.g., via a linker) to the second polynucleotide. In some embodiments, the immune cell is a T cell (e.g., cytotoxic T cell, helper T cell, regulatory T cell, gamma-delta T cell), an natural killer cell (NK cell), a macrophage, a tumor-infiltrating lymphocyte (TIL), a monocyte, or a B cell.

A pharmaceutical composition disclosed herein is prepared by per se known methods for the preparation of pharmaceutically acceptable compositions that are administered to subjects, such that an effective quantity of the immune cell is combined in a mixture with a pharmaceutically acceptable carrier. Suitable carriers are described, for example, in Remington's Pharmaceutical Sciences (Remington's Pharmaceutical Sciences, 20th ed., Mack Publishing Company, Easton, Pa., USA, 2000). On this basis, the compositions include, albeit not exclusively, solutions of the substances in association with one or more pharmaceutically acceptable carriers or diluents, and contained in buffered solutions with a suitable pH and iso-osmotic with the physiological fluids.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, N-(1(2,3-dioleyloxy)propyl)N,N,N-trimethylammonium chloride (DOTMA), diolesylphosphotidyl-ethanolamine (DOPE), and liposomes. In some embodiments, such compositions contain a therapeutically effective amount of the compound, together with a suitable amount of carrier so as to provide the form for direct administration to the patient.

Pharmaceutical compositions are administered in a manner appropriate to the disease to be treated (or prevented). The quantity and frequency of administration is determined by such factors as the condition of the patient, and the type and severity of the patient's disease, although appropriate dosages are determined by clinical trials. When “an immunologically effective amount,” “an anti-tumor effective amount,” “a tumor-inhibiting effective amount,” or “therapeutically effective amount” is indicated, the precise amount of the compositions of the present invention to be administered is determined by a physician with consideration of individual differences in age, weight, tumor size, extent of infection or metastasis, and condition of the patient (subject).

The pharmaceutical composition is “substantially free of” indicates, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium a fungus, mycoplasma, IL-2, and IL-7.

In some embodiments, the pharmaceutical compositions are administered in a single time or multiple times, for example, daily, weekly, biweekly, or monthly, hourly, or the pharmaceutical composition is administered upon recurrence, relapse or progression of the cancer being treated.

In some embodiments, a pharmaceutical composition disclosed herein is administered by any suitable method, including, without limitation, intravenously or by infusion. In some embodiments, a pharmaceutical composition disclosed herein is administered using infusion techniques that are commonly known in immunotherapy. In some embodiments, a pharmaceutical composition disclosed herein is injected directly into a tumor, lymph node, or site of infection.

### EXAMPLES

The following non-limiting examples are illustrative of the present application:

### Example 1. Chimeric Costimulatory Receptor

As stated previously, costimulation and coinhibition operate in parallel with TCR signaling to regulate T cell activation, proliferation and persistence. Modulating T cell responses via TNFRSF signaling is attractive for T cell therapies as each receptor confers unique biological effects. Moreover, given the similarity in the structural biology of the TNFRSF, it should be possible to re-direct coinhibition (i.e. suppressive signaling) by TNFRSF toward costimulation (i.e. stimulatory signaling) through the creation of chimeric costimulatory receptors (CCRs) based on TNFRSF. Indeed, previous reports have established that TNFRSF members have modular domains that can be swapped to redirect signaling.

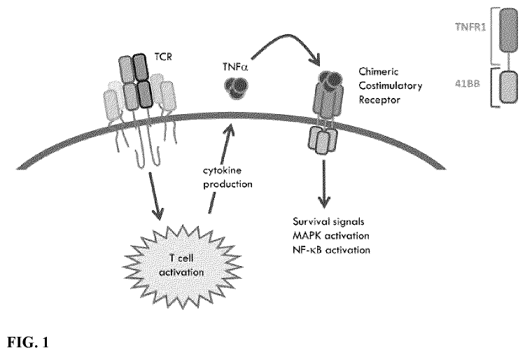

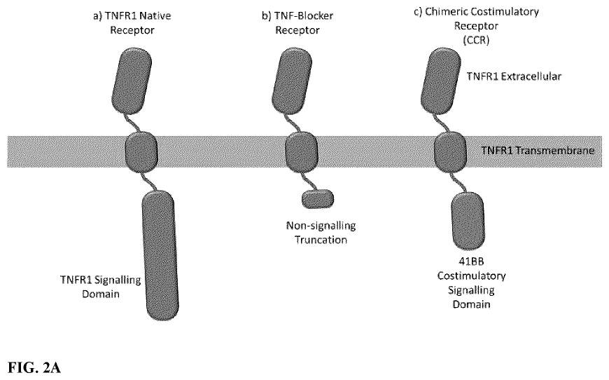

Anti-tumor T cells produce high levels of TNFα, yet experimental data suggests that natural TNFα signaling limits T cell immunity. TNFR1-mediated TNFα signaling causes cell death limiting expansion and function of T cells. Thus, it is of interest to consider redirecting TNFα biology through a chimeric TNFR1 that enhances T cell function following the elaboration of TNFα in response to antigen stimulation. Redirecting TNFR1 signaling towards T cell stimulation could be accomplished through switching the TNFR1 intracellular signaling domain for a costimulatory domain from another TNFRSF. In principle, the stimulus for such a chimeric costimulatory receptor (CCR) would be delivered in autocrine and paracrine through TNFα produced by T cells following recognition of cognate antigen; although, it is also possible that TNFα could be produced by other cells in the local milieu (e.g., macrophages). Recent success with costimulatory domains in the CAR field points to the costimulatory signaling domain of 4-1BB as having the most promising characteristics in T cells. CAR-engineered T cells bearing 4-1BB costimulatory domains display increase in memory markers, high persistence and resistance to anergy. Clinical trials utilizing 4-1BB CARs directed against the CD19 antigen demonstrated impressive proliferation upon infusion into the patient and persistence at high levels for 6 months. Thus, a CCR bearing the extracellular domain of TNFR1, which binds TNFα, joined to the cytoplasmic domain of 4-1BB, which provides costimulatory signaling, should provide robust proof-of-concept (FIG. 1). Of course, a TNFR1-based CCR would not be limited to the cytoplasmic domain of 4-1BB and the cytoplasmic domains of other TNFRSF, or combinations thereof, may provide even more robust outcomes.

To address the hypothesis that a TNFR1-4-1BB CCR could re-direct TNFα signaling, a fusion receptor was constructed comprised of the TNFα binding extracellular and transmembrane domains of the TNFR1 receptor joined to the intracellular signaling domains of 4-1BB (FIG. 2A). As a control, a TNF-Blocker receptor was also generated that would bind the cytokine but not transmit any signal (FIG. 2A).

Experimental Methods

Cloning

The full length TNFR1 coding sequence was ordered as a gBlocks gene fragment (IDT) and amplified by PCR to add restriction sites AscI and NheI to ligate into the pCCL transfer vector. Costimulatory TNFR1-4-1BB fusion was created through stitch PCR amplification using overlapping ends of TNFR extracellular domain and 4-1BB intercellular domain, added by PCR primers. Amplification of this product with primers to add restriction sites AscI and NheI allowed ligation into a pCCL transfer vector.

### Example 2. Characterization of the Chimeric Co-Stimulatory Receptor

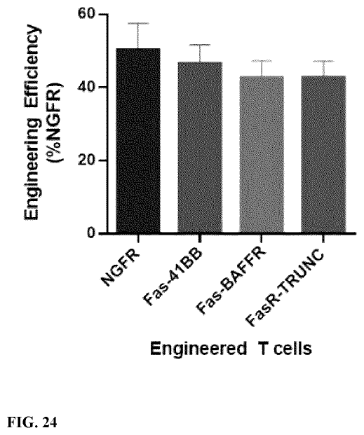

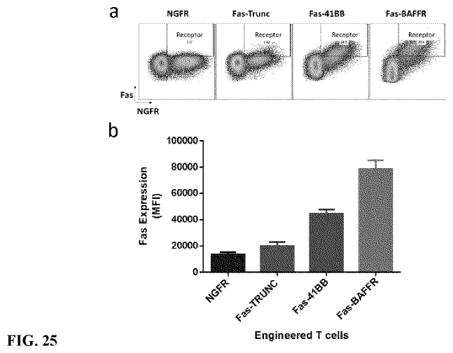

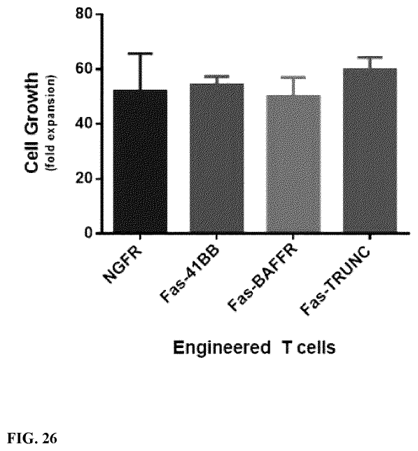

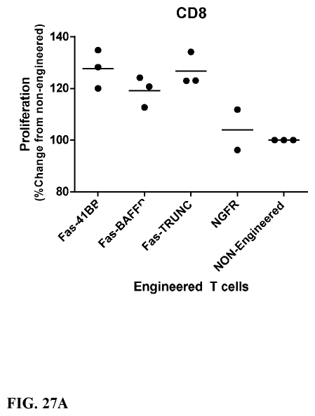

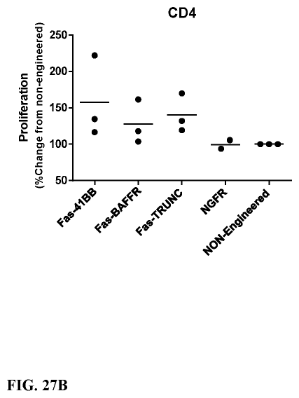

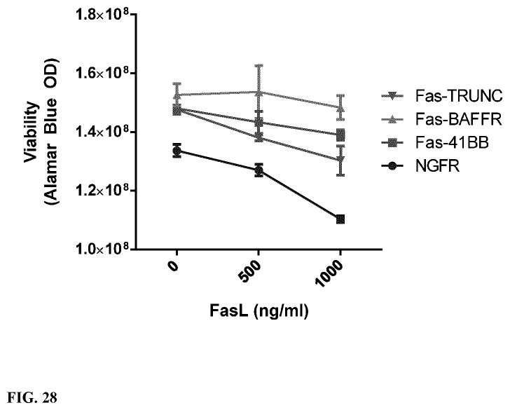

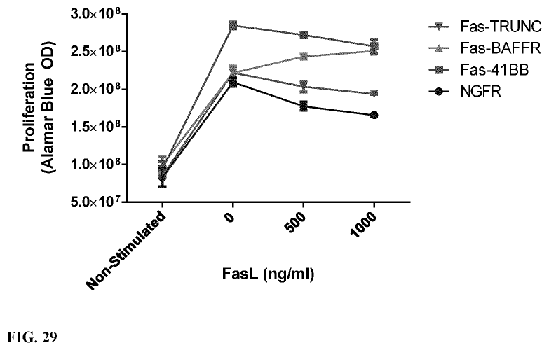

Activation of TNFRSF costimulatory domains results in the downstream activation of NFκB pathways to enhance the transcription of NFκB related genes including inflammation, survival and proliferation. To determine whether the TNFR1-4-1BB CCR enhances the transcription of NFκB-related genes, a reporter system was utilized where transcription of the firefly luciferase gene was controlled by two NFκB enhancer regions. HEK293TM cells were transfected, via Lipofectamine, with the NF-kB-driven luciferase reporter and one of: (i) full-length TNFR1, (ii) the TNF-Blocker, (iii) TNFR1-4-1BB CCR (FIG. 2A)). The transfected cells were subsequently stimulated with recombinant human TNFα. D-luciferin, the luciferase substrate, was added to the cultures and luminescence, a direct measure of luciferase abundance, was quantified by luminometry. These data show that expression of TNFR1-4-1BB CCR (FIG. 3, solid line) resulted in enhancement of NFκB transactivation relative to wild type TNFR1 (FIG. 3, large dashes) as demonstrated by an increase in luciferase reporter activity. Expression of the dominant negative TNF-Blocker (FIG. 2B) receptor abolishes NFκB signaling through TNFα (FIG. 3, short dashes). However, these data also suggest the TNFR1-4-1BB is constitutively active in HEK293TM cells as NF-kB transactivation was observed in the absence of TNFα.

Activation of the NFκB pathway results from a complex signaling cascade involving the activation of several kinases involved in mediating activation of the ubiquitin-proteasome pathway. A key inhibitor of NFκB signaling, Inhibitor of κB (IκB), sequesters important NFκB factors and upon NFκB activation is targeted for degradation via the ubiquitin-proteasome pathway. Degradation of IκB activates the NFκB transcription factors to enhance gene transcription. To determine whether activation of the CCR results in the degradation of IκB, western blots were performed. Since the apparent constitutive activation of the TNFR1-4-1BB CCR may be due to expression in HEK293TM cells, for these experiments, TNFR1 and the variant TNFR1 receptors were introduced into a T cell line, Jurkat, via lentivirus transduction. Upon stimulation with TNFα, the Jurkat cells transduced with the TNFR1-4-1BB CCR revealed time-dependent degradation of IκB; the rate of degradation was enhanced compared to parental non-engineered (WT) Jurkat cells demonstrating a potent effect of the TNFR1-4-1BB CCR (FIG. 4). The TNF-Blocker receptor with a truncated non-signalling cytoplasmic domain abrogated TNFα stimulated IκBα degradation (FIG. 4), indicating that the enhanced degradation of IκB by the TNFR1-4-1BB CCR was due to the cytoplasmic domain. Wild type TNFR1 could not be overexpressed in the Jurkat cells due to the toxic effects of the wild type TNFR1.

4-1BB signalling has been shown to activate the MAPK signalling pathway. Phosphorylation and activation of p38 MAPK leads to activation of downstream transcription factors. Phospho-p38 protein levels were assessed following TNFα stimulation of TNFR1-4-1BB CCR and TNF-Blocker engineered Jurkat cells. Stimulation with TNFα induced the induction of phospho-p38 levels in both non-engineered WT cells and those engineered with the TNFR1-41BB CCR. Phospho-p38 levels reached their peak at 5 min post stimulation. The peak response was higher in the Jurkat cells engineered with TNFR1-4-1BB CCR compared to non-engineered (WT) Jurkat cells (FIG. 5). Similar to the results observed with IκB, the expression of the TNF-Blocker receptor, the TNF-Blocker receptor abrogated p38 phosphorylation (FIG. 5).

These results clearly demonstrate that the TNFR1-4-1BB CCR is capable of redirecting TNFα signalling towards enhanced NF-kB signalling in T cells.

A third-generation self-inactivating lentiviral expression system was used to engineer primary T cells to stably express one of the receptors in FIGS. 2A-C. Transduced T cells were evaluated for surface expression of the receptor via staining and flow cytometry. Expression of the TNFR1-4-1BB CCR was evident in both CD8+ and CD4+ T cell subsets with higher transduction rates noted in CD4+ T cells (FIGS. 6A-B). Following activation and transduction of T cells, the growth of primary cultures was followed over 14 days. Expression of the TNFR1-4-1BB CCR in T cells did not impact the growth of cells in vitro suggesting transduced T cells tolerated the introduction of the TNFR1-4-1BB CCR (FIG. 7).

TNFRSF costimulation has been shown to enhance survival signaling in T cells to improve persistence. 4-1BB costimulation provided by the TNFR1-4-1BB CCR is expected to provide enhanced survival characteristics to transduced T cells. Primary human T cells were transduced with the TNFR1-4-1BB CCR and grown in IL2 and IL7 growth factors for 14 days. On day 14, cells were removed from cytokine and stimulated with either signal 1 (Anti-CD3) or signal 2 (TNFα) and live cell numbers were followed for 4 days. Removal of growth factors from T cells results in the apoptosis and death of control T cell cultures. However, T cells transduced with the TNFR1-4-1BB CCR demonstrated better survival in both stimulation conditions (FIGS. 8A-B), suggesting that TNFα stimulation of the TNFR1-4-1BB CCR enhanced survival signaling to engineered T cells upregulating anti-apoptotic pathways. These data also suggest that anti-CD3 stimulated T cells secrete a sufficient level of TNFα to stimulate TNFR1-4-1BB CCR survival signaling.

TNFR1-4-1BB CCR transduced T cells stimulated with signal 1 (Anti-CD3) become activated to secrete both TNFα and IFNγ. The cytokine profile of CCR T cells is biased towards IFNγ production compared to control cells (FIG. 9).

Given the promising function of the TNFR1-4-1BB CCR, it was then determined whether expression of the CCR would provide costimulatory activity in the context of T cell activation via a synthetic antigen receptor. To this end, the CCR was co-expressed with a TAC receptor specific for BCMA (BCMA-TAC). Lentiviruses were constructed that expressed the TAC receptor with either the TNFR1-4-1BB CCR or the TNF-Blocker separated by a 2A peptide (FIG. 10).

To ensure that the expression of BCMA-TAC and TNFR1-4-1BB CCR under a 2A expression system does not interfere with TAC functionality, functional assays to assess anti-tumor activity were performed. TAC and TAC+CCR engineered T cells were co-cultured for 24 hrs with BCMA-positive KMS11 cells expressing luciferase. Killing activity was measured by the reduction in luminescence of the KMS11 targets. TAC+CCR T cells demonstrate similar killing activity to TAC T cells (FIG. 11) indicating the 2A expression system results in functional receptor expression. Since T cell activation and cytotoxicity are mediated through the TAC receptor alone, the additional signals provided by the 4-1BB-CCR were not expected to influence in-vitro killing assays.

Activated T cells mediate cytotoxicity and pro-inflammatory signaling through several soluble mediators including secreted cytokines. The expression of IFNγ and TNFα were assessed in an in vitro intracellular cytokine assay following co-culture with antigen positive tumor cell targets. Upon antigen engagement both TAC− and TAC+CCR-engineered T cells expressed IFNγ and TNFα. It is interesting that there was an observed reduction in cytokine expression in both CD4+ and CD8+TAC+CCR T cells (FIG. 12). In the clinic, the use of CAR engineered T cells exhibits a high risk of toxicity to the patient, mediated in part through a cytokine storm. The reduction of T cell secreted cytokines following antigen engagement alongside expression of the CCR may be an effective means of mitigating CART cell toxicity while maintaining anti-tumor activity.

To determine the functional outcome of CCR expression in TAC engineered T cells, proliferation assays were performed to track T cell division following co-culture with antigen positive tumor targets. Upon antigen engagement and T cell activation, T cells undergo rapid division and proliferation, producing daughter cells to mediate anti-tumor activity. T cells engineered with TAC alone, TAC+CCR and TAC+TNF-Blocker were compared in a proliferation assay. Following a seven-day proliferation assay, both CD4+ and CD8+TAC T cells engineered with the 4-1BB-CCR displayed enhanced proliferation (FIGS. 13A-B). The effect is more pronounced in CD4+ compared to CD8+ T cells. T cells engineered with the TNF-Blocker negatively impacted proliferation as compared to TAC alone. The data suggests that expression of CCR is mediating signals to enhance proliferative capacity of TAC T cells. Comparing the Proliferation and Division index between TAC and TAC+CCR engineered, it was observed that TAC+CCR enhances the proliferation of TAC T cells. These values also reveal that, on average, TAC+CCR generates 30-50% more proliferating cells than TAC T cells alone.

The TNFRSF contains several molecules known to co-stimulate T cells, including 4-1BB, OX40 and CD27 capable of enhancing proliferation, survival and memory development. Given the promising results with the prototypic TNFR1-4-1BB CCR, it was sought to determine whether other costimulatory domains would have equivalent, or greater, activity. Alternate TNFRSF costimulatory signals may induce TAC engineered T cells with unique characteristics to improve anti-tumor activity. 18 intracellular domains were selected to evaluate (listed in Table 1) and a series of TNFRSF CCRs were designed where the transmembrane domain was derived from TNFR1 or the respective TNFRSF (as shown in Table 1).

To screen the utility of TNFRSF CCRs, the CCRs were evaluated for their ability to enhance proliferation of the engineered T cells following stimulation with plate-bound anti-CD3 (OKT3).

To confirm the utility of the screen, T cells were engineered with the original TNFR1-4-1BB, the TNF-Blocker, or a control receptor (truncated NGFR) and activated with plate bound anti-CD3 (FIG. 14). Consistent with prior data, the screen identified a proliferative enhancement by the TNFR1-4-1BB CCR and an inhibitory effect of the TNF-Blocker, relative to the control (truncated NGFR). Thus, plate-bound CD3 is a valid assay for screening of the TNFRSF costimulatory receptors.

The cDNAs for the synthetic TNFR1-CCRs described in Table 1 were synthesized by GenScript and subcloned into lentiviral vectors. PBMCs stimulated with anti-CD3/anti-CD28 beads were lentiviral engineered with CCR and grown in IL2 and IL7 for 14 days at which point, the T cells assessed for overall growth, engineering efficiency, receptor expression, and proliferation following stimulation with plate-bound anti-CD3. Engineering efficiency of human PBMCs with the CCRs ranged from 15-85%, as assessed by the transduction marker (NGFR) included in all of the lentiviruses (FIG. 15). Engineered cells were stained with antibody against the extracellular TNFR1 domain to evaluation of amount of receptor on the surface. Surface expression of CCRs ranged from 0-1500% above background (FIG. 16). Fold expansion of the bulk CCR engineered cultures ranged from 50-450% of control (FIG. 17). Day 14-engineered T cells were evaluated in a 5-day proliferation assay following anti-CD3 stimulation. The proliferation of CCR engineered T cells ranged from 1 (no proliferation) to 15 (on average 15 cells generated from a single cell) in both CD4 (FIG. 18) and CD8 (FIG. 19) T cell subsets. The positive hit threshold for the proliferation screen of CCRs was set at the proliferation index of NGFR transduction control, which received no costimulatory signals. The proliferation of CD4 T cells engineered with CCRs demonstrated a more pronounced effect on proliferation than CD8 T cells. The most proliferative CCR CD4 cells were upwards of 75% more proliferative than NGFR engineered cells, whereas the most proliferative CCR CD8 T cells were 60% more proliferative than corresponding NGFR engineered cells.

The multifactorial screening results from the designed constructs were analyzed using a clustering algorithm to assess the similarities between CCR engineered T cells. Data included in the clustering algorithm were surface expression, engineering efficiency, and proliferation of both CD4+ and CD8+ T cell subsets. Data dimensionality was first reduced using Principle Component Analysis and centroids were calculated using a K-means clustering method. A correlation heat map describes the similarity between constructs (FIG. 20). The groupings of the dendrogram identify receptors with similar attributes. The upper group contains 4-1BB and BAFFR CCRs that have demonstrated higher surface expression, improved growth of cultures, and enhanced proliferation. The middle grouping contains the NGFR transduced control with no CCR expression, minimal impact on growth, no enhancement to proliferation. The middle group also contains the original 4-1BB CCR which has little CCR expression, minimal improvement to growth, and some enhancement to proliferation. The lower group contains the TNF-Blocker, LIGHT, and FAS costimulatory domains, which have been demonstrated to slow culture growth, and negatively impact proliferation. It is intriguing that these groups were formed based on the multiparameter clustering, which provides insight into the costimulatory domains and highlights the members in the upper group that are clustered together with positive hits from the proliferation screen. Although not all receptors in this group provided enhancement to proliferation, they may still be worth pursuing. Costimulatory receptors not only enhance proliferation but also enhance cytotoxic function and survival and persistence. The receptors in this costimulatory grouping may have potential for other costimulatory properties beside proliferation.

Evaluating the groupings of TNFRSF CCRs created from clustering of centroids revealed engineered T cells with desirable functional attributes. CCRs with the 4-1BB and BAFFR costimulatory signaling domains demonstrated improved expansion of PBMCs, high engineering efficiency, and improved proliferative capacity. To validate the utility the 4-1BB and BAFFR domains, the TNFR1-4-1BB and TNFR-BAFFR constructs were run in a proliferation assay with 3 PBMC donors (FIGS. 21A-B). The selected constructs performed comparably to the screen results, demonstrating enhanced proliferation over the control receptors. These data provided confidence in the screen data as well as confirm the potential in using the 4-1BB and BAFFR signaling domains for the costimulation of engineered T cells. An important function of cytotoxic T cells is the secretion of cytokines following activation. TNFR1-4-1BB and TNFR-BAFFR engineered T cells were evaluated for cytokine production following anti-CD3 stimulation (FIGS. 22A-B) where comparable levels of IFN-γ-producing cells were observed, but diminished levels of TNF-α producing cells were observed, presumably because the TNF-α is binding to the CCR.

### DISCUSSION

T cell activation is the result of a complex, well-orchestrated signaling cascade. Full recruitment of the TCR complex, coreceptors, adaptor molecules as well as integration of costimulatory and coinhibitory molecules results in the summation of receptor signal strength leading to induction of activated T cell programming. The TCR is a critical component of this circuit. The TCR directs the focus of T cell cytotoxicity towards a unique epitope of a pathogen. The development of synthetic antigen receptors, including the T cell Antigen Couplers (TAC), has led to the ability to direct the cytotoxic genius of a T cell towards novel targets; repurposing the human immune system with enhanced tumor surveillance and anti-tumor activity. TAC molecules deliver signal-1 of the two-signal hypothesis. Engaging signal-2 within engineered cells may induce a stronger stimulus to T cell activation. The success of T cell-activating chimeric receptors imagines the idea of developing novel chimeric costimulatory receptors to induce unique characteristics in engineered cells. Costimulatory receptors including CD28, 4-1BB and OX40 are well described receptors that provide second signals to activated T cells promoting activation, proliferation and cytotoxic function. As described above, a chimeric costimulatory receptor (CCR) capable of providing costimulation under the control of an activated T cell secreted ligand TNFα was developed. The inspiration behind this design methodology was to provide costimulation only following T cell activation. These studies have found that activated T cells secret abundant TNFα. In principle, the secreted TNFα should bind and stimulate the CCR. The initial CCR construct design contained the costimulatory molecule of the TNFRSF, 4-1BB. The CCR engineers the costimulatory signal as a separate receptor resulting in its utility across many cell therapies, including all forms of T cell therapy (e.g., engineered T cells, TILs), NK cell applications and engineered monocyte therapies. The CCR is this project displays enhanced proliferative capacity and altered cytokine production both when expressed alone and in combination with the TAC receptor. The enhanced proliferation observed in CCR engineered T cells may provide the adaptive edge required to overcome the suppressive microenvironment of entrenched tumor masses, which continues to present challenges to the field of adoptive T cell transfer. The altered cytokine production profile of CCR T cells appears to result in no impairment to proliferation, cytotoxicity or survival. Reduced cytokine production may even prove to be less toxic to patients following administration of engineered T cells. Currently, the infusion and expansion of CAR T cells in patients results in toxicity stemming from a cytokine storm that may be abated with engineering T cells with altered cytokine production profiles.

The improvement in T cell proliferation found when including a 4-1BB CCR lead to an analysis of the utility of other costimulatory domains derived from the broad family of TNFRSF for costimulation of engineered T cells. The proliferation screen of TNFRSF costimulatory signaling domains within the TNFR1-chimera platform revealed a variety of properties of the CCR with potential for enhancing T cell function. Of note, this screen revealed the utility of the BAFF-R costimulatory domain, which is not typically thought of as a costimulator of T cells. Importantly, the utility of these various CCRs may differ depending upon the application, which creates value for the entire collection of CCRs that were generated. The aim is to generate a TAC+CCR T cell product with enhanced in vivo anti-tumor activity. Future directions will include continuing to evaluate the effect of CCR on engineered T cells. Indeed, the outcomes of these data suggest that the CCR design described herein can be used to re-direct signaling through any of the TNFRSF that mediate inhibitory and/or death signals to T cells. As examples of further iterations, CCRs that employ the extracellular domain of FAS (TNFRSF6) to disrupt death signaling from FasL (TNFSF6) or DR4/DR5 (TNFRSF10A/B) to disrupt death signaling from TRAIL (TNFSF10) as both FasL and TRAIL can promote T cell death following activation may be designed. CCR engineered T cells have been demonstrated to have enhanced proliferation; future work will determine the cytokine production profile of these costimulated T cells. It will be interesting to investigate the gene pathways altered by CCR signaling and discover the regulators of proliferation in human T cells.

Experimental Methods

T Cell Growth

Peripheral blood mononuclear cells are received from healthy donors and stimulated with anti-CD3 and anti-CD28 magnetic beads in RPMI culture media supplemented with IL-2 (100 U/ml) and IL-7 (long/ml). After 1 day, cells were transduced using a third-generation lentiviral vector and packaging system. T cell cultures are maintained at 1×10⁶ cells/ml with the addition of IL-2 and IL-7 every two days. After 14 days in culture, T cells are characterized for CD4/CD8, chimeric receptors, NGFR (transduction marker).

Lentiviral Production

Lentivirus were prepared by transfection of HEK293T cells with the packaging plasmids pRSV-Rev, pMDLg-pRRE, pMD2.G, and the pCCL transfer plasmid by Lipofectamine 2000 transfection reagent (Life Technologies). Particles were concentrated by ultracentrifugation at 28,000 RPM. Viral titre were determined by dilution of virus and transduction of HEK293T cells. Transduced HEK293T cells were quantified for % NGFR+(transduction marker) by flow cytometry and titre calculated in transduction units (TU/ml).

Determining Protein Level (Western Blot)

T cells were homogenized on ice using a tissue homogenizer in lysis buffer (150 mM NaCl, 0.5% sodium deoxycholate, 0.1% SDS, 50 mM Tris, 1% Triton X100, pH 8.0) with protease inhibitor cocktail (1:100) (Sigma Aldrich), followed by centrifugation at 9,000×g for 15 min at 4° C. Sample protein concentrations was determined by bicinchoninic acid assay (Sigma Aldrich). Laemmli buffer with 2-mercaptoethanol was added to samples followed by heat denaturing at 95° C. for 5 min. Samples were electrophoresed through 10% SDS-PAGE gels and wet-transferred to nitrocellulose membranes at a constant 350 mA for 90 min. Membranes were blocked overnight at 4° C. with 5% (w/v) skim milk blocker in TBST. Membranes were incubated, shaking for 2 h at room temperature with primary antibodies (1:1000 in TBST with 2% blocker). Membranes were washed 3 times in TBST and incubated for 1 h with fluorescent-conjugated secondary antibodies (1:5000 in TBST with 2% skim milk blocker). Membranes were washed 3 times with TBST.

Flow Cytometry Surface Staining

T cells were harvested, pelleted at 1500 rpm and incubated for 30 min with fluorescent-label antibodies against plasma membrane proteins. T cells were washed in FACS buffer (1% BSA, 1×PBS, 2.5 mM EDTA) and pelleted. T cells were suspended in 300 μl FACS buffer and filtered before running on the flow cytometer. The flow cytometer records forward scatter, side scatter and appropriate fluorescent channels for the labeled antibodies. Cytometer data were analyzed using FlowJo V10 software and displayed as scatter plots.

In Vitro Intracellular Cytokine Production

T cells were harvested and stimulated with antigen positive tumor cell lines for 4 hrs at 37° C. GolgiPlug and GolgiStop reagents (BD Bioscience) were added to prevent T cell secretion of cytokines. After 4 hrs, the stimulation is stopped with addition of 0.02M EDTA and incubated at room temperature for 15 min. Cells were collected and washed followed by centrifugation and staining. Intracellular cytokines are assessed by fixing and permeabilizing the cell followed by staining with fluorescent-labelled antibodies against TNF-alpha, IFN-gamma, and IL-2. Fluorescence is assessed by flow cytometry and analyzed on FlowJo V10 software.

In Vitro Cytotoxicity

T cells were harvested and co-incubated with luciferase-expressing antigen-positive tumor cell lines at an effector to target ratio from 8 to 0.25 for 16-18 hrs. Following incubation, D-luciferin is added to the culture and incubated for 10 min at room temperature. Luminance is read on a plate reader. The amount of luminance correlates to the viability of tumor cells in a well. Data is plotted across effector to target ratio.

Proliferation Assay

T cells were harvested, pelleted at 1500 rpm and resuspended in 1:1000 CellTrace™ Violet stain in PBS for 20 min at 37° C.; staining cells at 1×10⁶ cells/ml. T cell media is added at 4:1 the volume of stain and incubated at 37° C. to quench the remaining CellTrace™ dye. Stained cells are pelleted at 1500 rpm and resuspended in T cell media for plating at 0.5×10⁶ cells/ml in a 24 well plate with no cytokine. Tumor targets are added to the wells at 1:2 effector:target ratio. For single expression of CCR, cells are stimulated with plates coated with 10 μg/ml Anti-CD3 (OKT3 clone). T cells are followed for either 5 or 7 days, recording cell number and viability. On day 5 or 7 cells are harvested and stained for flow cytometry. The CellTrace™ Violet dilution peaks represent divided T cells and from this a measure of proliferation can be calculated.

### Example 3: Fas Chimeric Costimulatory Receptors

The TNFRSF, Fas or CD95, is an important regulator of T cell apoptosis.

Expression of FasL has been demonstrated on T cells and tumors. Fas-FasL interaction can promote the death of activated T cells via the interaction of FasL-expressing T cells and Fas-expressing T cells resulting in fratricide. Interaction between FasL on tumors and Fas-expressing T cells has also been linked to cell death of tumor-specific T cells. The death signal is mediated via the cytoplasmic domain of the Fas receptor which interacts with the adapter protein, FADD or Fas-associated protein with death domain, which recruits caspase-8 and caspase-10 to form the death inducing signalling complex (DISC). DISC cleaves and activates caspase-8/caspase-10 to trigger effector caspases to mediate apoptosis.

Removal of the cytoplasmic region of Fas would result in a dominant negative receptor that would be expected to attenuate the cell death signal. Replacement of the Fas cytoplasmic domain with the cytoplasmic domain of either 4-1BB or BAFFR would be expected to squelch the death signal and replace it with a costimulatory signal that could enhance T cell survival.

Fas chimeras were produced where the extracellular domain of the Fas receptor was joined to either the cytoplasmic domain of 4-1BB or BAFFR as shown in FIG. 2B.

Engineering and Expression of Fas-Chimeric Costimulatory Receptors

Primary human T cells were transduced with lentiviruses expressing the Fas-Chimeras or Fas-TRUNC. As a control, primary human T cells were transduced with a control lentivirus that encoded no Fas receptor (NGFR). All the lentivirus vectors expressed NGFR enabling the determination of transduction efficiency based on the expression of NGFR. All constructs for engineered T cells displayed an efficiency between 40-50% based on NGFR transduction marker expression, which was comparable to the control virus (FIG. 24).

Fas-chimeras were detected on the cell surface with antibodies against the native Fas. As noted above, non-engineered T cells express high levels of Fas on day 14 of the culture period. T cells transduced to express the Fas-chimeras containing the 4-1BB and BAFFR costimulatory domains demonstrated Fas expression levels, as evaluated by mean fluorescent intensity, above that of native Fas levels (FIG. 25). Cell surface expression of the Fas-4-1BB and Fas-BAFFR chimeras, as evaluated by mean fluorescent intensity, were 4.9×10⁴ and 7.9×10⁴ compared to 1.35×10⁴ in the NGFR control. Interestingly, the Fas-TRUNC receptor was barely expressed above NGFR levels (MFI=2.0×10⁴).

T cells engineered to express the Fas-chimeras grew at comparable rates during the manufacturing process, compared to NGFR control, indicating that the expression of the modified Fas receptors do not influence T cell growth during the manufacturing period (FIG. 26).

Modified Fas Receptors Enhance Proliferation

To determine whether expression of the modified Fas receptors influence T cell proliferation, primary human T cells were engineered with lentiviruses expressing either Fas-TRUNC, Fas-4-1BB, or Fas-BAFFR. Control T cells were engineered with a lentivirus that expressed only NGFR. The engineered T cells were stimulated with an agonist CD3 antibody as a surrogate stimulus for the T cell receptor. All of the T cells engineered with the modified Fas receptors displayed enhanced proliferation relative to the control NGFR T cells. T cells engineered with Fas-4-1BB or Fas-BAFFR proliferated equally to the Fas-TRUNC, indicating that squelching the Fas signal was sufficient to enhance proliferation without requiring additional costimulatory signaling (FIG. 27).

Fas-Chimeras Overcome Exogenous FasL Signaling

The Fas-chimeras were designed to redirect the apoptotic signal of FasL. To determine whether Fas-chimeras can block apoptosis from FasL, engineered T cells were stimulated with soluble trimeric FasL and viability was assessed 48 hrs later using the metabolic dye, AlamarBlue. Exposure of control cells to FasL produced reduced T cell viability with increasing concentration. Control T cells (NGFR) displayed dose-dependent loss in viability in the presence of exogenous FasL. All T cells engineered with modified Fas receptors displayed enhanced viability compared to control cells in the presence of exogenous FasL. The T cells engineered with Fas-BAFFR displayed the greatest resistance to exogenous FasL indicating a benefit of the costimulatory domain to T cell survival (FIG. 28).

Fas-Chimera Enhances Proliferation in the Presence of FasL

As T cells engineered with the modified Fas receptors were protected from FasL mediated cell death following cytokine withdrawal, it was sought to determine the impact of exogenous FasL on the proliferation of T cells engineered with the modified Fas receptors. T cells engineered with the modified Fas receptors with activated with anti-CD3 and cell density was assessed via AlamarBlue 3-days later. The presence of exogenous FasL resulted in lower density of control T cells (FIG. 29). A similar decline in cell density was observed with T cells engineered with the Fas-TRUNC receptors. However, T cells engineered to express Fas-chimeras containing costimulatory signalling domains demonstrated enhanced proliferation in the presence of 500 and 1000 ng/ml FasL compared to NGFR control engineered T cells (FIG. 29). T cells engineered to express Fas-BAFFR that demonstrated an ability to enhance proliferation with increasing concentrations FasL, whereas T cells engineered with Fas-4-1BB were not influenced by exogenous FasL(FIG. 29).

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
TNFRSF CCR Construct Design
Costimulatory Screen

| | | | | Death |
|---|---|---|---|---|
| Construct | TNFRSF# | Transmembrane | Costim | Domain |
| 1 | TNFRSF3 | TNFR1 | LIGHT | No |
| 2 | TNFRSF3 | LIGHT | LIGHT | No |
| 3 | TNFRSF4 | TNFR1 | OX40 | No |
| 4 | TNFRSF4 | OX40 | OX40 | No |
| 5 | TNFRSF5 | TNFR1 | CD40 | No |
| 6 | TNFRSF5 | CD40 | CD40 | No |
| 7 | TNFRSF6 | TNFR1 | Fas | Yes |
| 8 | TNFRSF6 | Fas | Fas | Yes |
| 9 | TNFRSF7 | TNFR1 | CD27 | No |
| 10 | TNFRSF7 | CD27 | CD27 | No |
| 11 | TNFRSF8 | TNFR1 | CD30 | No |
| 12 | TNFRSF8 | CD30 | CD30 | No |
| 13 | TNFRSF9 | TNFR1 | 41BB | No |
| 14 | TNFRSF9 | 41BB | 41BB | No |
| 15 | TNFRSF11A | TNFR1 | RANK | No |
| 16 | TNFRSF11A | RANK | RANK | No |
| 17 | TNFRSF12A | TNFR1 | TWEAK | No |
| 18 | TNFRSF12A | TWEAK | TWEAK | No |
| 19 | TNFRSF13b | TNFR1 | TACI | No |
| 20 | TNFRSF13b | TACI | TACI | No |
| 21 | TNFRSF13C | TNFR1 | BAFFR | No |
| 22 | TNFRSF13C | BAFFR | BAFFR | No |
| 23 | TNFRSF14 | TNFR1 | HVEM | No |
| 24 | TNFRSF14 | HVEM | HVEM | No |
| 25 | TNFRSF18 | TNFR1 | GITR | No |
| 26 | TNFRSF18 | GITR | GITR | No |
| 27 | TNFRSF19 | TNFR1 | TROY | No |
| 28 | TNFRSF19 | TROY | TROY | No |
| 29 | TNFRSF19I | TNFR1 | RELT | No |
| 30 | TNFRSF19I | RELT | RELT | No |
| 31 | TNFRSF1B | TNFR1 | TNFR1B | No |
| 32 | TNFRSF1B | TNFR1B | TNFR1B | No |
| 33 | TNFRSF25 | TNFR1 | DR3 | Yes |
| 34 | TNFRSF25 | DR3 | DR3 | Yes |
| 35 | TNFRSF27 | TNFR1 | XEDAR | No |
| 36 | TNFRSF27 | XEDAR | XEDAR | No |

Table 1 depicts the construct design for the Chimeric Costimulatory Receptor screen. The TNFR-fusion receptors demonstrated functional enhancements to engineered T cells in vitro, thus it is attractive to pursue novel costimulatory domains within the TNFR Superfamily. The costimulatory screen is designed to evaluate T cell costimulatory function of fusions between TNFR1 extracellular domain with the costimulatory signaling domain of TNFRSF members. The table describes the transmembrane and costimulatory domain used for each construct in the screen. The table also lists whether the designed CCR contains a classical death domain known for signaling a caspase cascade.

1. A Chimeric Costimulatory Receptor (CCR) nucleic acid, comprising (a) a first polynucleotide encoding an extracellular domain of a member of the Tumor Necrosis Factor Receptor Superfamily; (b) a second polynucleotide encoding a transmembrane domain polypeptide; and (c) a third polynucleotide encoding a cytosolic costimulatory signaling domain polypeptide.

2. The Chimeric Costimulatory Receptor (CCR) nucleic acid of claim 1, wherein the first polynucleotide and the third polynucleotide are derived from different members of the TNFRSF.

3. The Chimeric Costimulatory Receptor (CCR) nucleic acid of claim 1, wherein the first polynucleotide encodes an extracellular domain of TNFRSF member having a death domain.

4. The Chimeric Costimulatory Receptor (CCR) nucleic acid of claim 1, wherein the first polynucleotide encodes an extracellular domain of TNFR1, TNFR2, Fas, DR4, DR5, DR3, DR6, EDAR, XEDAR, TROY or NGFR.

5. The Chimeric Costimulatory Receptor (CCR) nucleic acid of claim 1, wherein the first polynucleotide encodes an extracellular domain of TNFR1.

6. The Chimeric Costimulatory Receptor (CCR) nucleic acid of claim 1, wherein the first polynucleotide encodes an extracellular domain of TNFR2.

7. The Chimeric Costimulatory Receptor (CCR) nucleic acid of claim 1, wherein the first polynucleotide encodes an extracellular domain of Fas.

8. The Chimeric Costimulatory Receptor (CCR) nucleic acid of claim 1, wherein the first polynucleotide encodes an extracellular domain of DR4.

9. The Chimeric Costimulatory Receptor (CCR) nucleic acid of claim 1, wherein the first polynucleotide encodes an extracellular domain of DR5.

10. The Chimeric Costimulatory Receptor (CCR) nucleic acid of claim 1, wherein the first polynucleotide encodes an oligopeptide at least 80%, at least 85%, at least 90%, at least 95%, at least 99% or 100% identical to SEQ ID NO: 1.

11. The Chimeric Costimulatory Receptor (CCR) nucleic acid of claim 1, wherein the first polynucleotide encodes an oligopeptide at least 80%, at least 85%, at least 90%, at least 95%, at least 99% or 100% identical to SEQ ID NO: 2.

12. The Chimeric Costimulatory Receptor (CCR) nucleic acid of claim 1, wherein the third polynucleotide encodes a cytosolic costimulatory signaling domain from a Tumor Necrosis Factor Receptor Superfamily (TNFRSF) member.

13. The Chimeric Costimulatory Receptor (CCR) nucleic acid of claim 1, wherein the third polynucleotide encodes a cytosolic costimulatory signaling domain from 4-1BB, BAFFR, OX40, CD27, CD40, GITR, HVEM, OX40, RELT, TACI, TROY, or TWEAK.

14. The Chimeric Costimulatory Receptor (CCR) nucleic acid of claim 1, wherein the third polynucleotide encodes a cytosolic costimulatory signaling domain from BAFFR.

15. The Chimeric Costimulatory Receptor (CCR) nucleic acid of claim 1, wherein the third polynucleotide encodes a cytosolic costimulatory signaling domain from 4-1BB.

16. The Chimeric Costimulatory Receptor (CCR) nucleic acid of claim 1, wherein the third polynucleotide encodes a cytosolic costimulatory signaling domain from CD27.

17. The Chimeric Costimulatory Receptor (CCR) nucleic acid of claim 1, wherein the third polynucleotide encodes a cytosolic costimulatory signaling domain from HVEM.

18. The Chimeric Costimulatory Receptor (CCR) nucleic acid of claim 1, wherein the third polynucleotide encodes a cytosolic costimulatory signaling domain from OX40.

19. The Chimeric Costimulatory Receptor (CCR) nucleic acid of claim 1, wherein the third polynucleotide encodes a cytosolic costimulatory signaling domain from GITR.

20. The Chimeric Costimulatory Receptor (CCR) nucleic acid of claim 1, wherein the third polynucleotide encodes a cytosolic costimulatory signaling domain from TACI.

21. The Chimeric Costimulatory Receptor (CCR) nucleic acid of claim 1, wherein the third polynucleotide encodes an oligopeptide at least 80%, at least 85%, at least 90%, at least 95%, at least 99% or 100% identical to SEQ ID NO: 3.

22. The Chimeric Costimulatory Receptor (CCR) nucleic acid of claim 1, wherein the third polynucleotide encodes an oligopeptide at least 80%, at least 85%, at least 90%, at least 95%, at least 99% or 100% identical to SEQ ID NO: 4.

23. The Chimeric Costimulatory Receptor (CCR) nucleic acid of claim 1, wherein the transmembrane domain polypeptide is the transmembrane domain polypeptide from a member of the Tumor Necrosis Factor Receptor Superfamily.

24. The Chimeric Costimulatory Receptor (CCR) nucleic acid of claim 1, wherein the second polynucleotide encodes an oligopeptide at least 80%, at least 85%, at least 90%, at least 95%, at least 99% or 100% identical to SEQ ID NO: 5.

25. The Chimeric Costimulatory Receptor (CCR) nucleic acid of claim 1, wherein the second polynucleotide encodes an oligopeptide at least 80%, at least 85%, at least 90%, at least 95%, at least 99% or 100% identical to SEQ ID NO: 6.

26. The Chimeric Costimulatory Receptor (CCR) nucleic acid of claim 1, wherein the transmembrane domain and cytosolic costimulatory signaling domain are from the same costimulatory signaling protein.

27. The Chimeric Costimulatory Receptor (CCR) nucleic acid of claim 1, wherein the transmembrane and costimulatory signaling domains are derived from 4-1BB, BAFFR, OX40, CD27, CD40, GITR, HVEM, OX40, RELT, TACI, TROY, or TWEAK.

28. The Chimeric Costimulatory Receptor (CCR) nucleic acid of claim 1, wherein the first polynucleotide and second polynucleotide are joined directly and/or indirectly to the third polynucleotide.

29. The Chimeric Costimulatory Receptor (CCR) nucleic acid of claim 1, wherein the first polynucleotide and third polynucleotide are joined directly and/or indirectly to the second polynucleotide.

30. The Chimeric Costimulatory Receptor (CCR) nucleic acid of claim 1, wherein the first polynucleotide and/or the second polynucleotide are joined indirectly to the third polynucleotide by a linker.

31. The Chimeric Costimulatory Receptor (CCR) nucleic acid of claim 1, wherein the first polynucleotide and/or the third polynucleotide are joined indirectly to the second polynucleotide by a linker.

32. A Chimeric Costimulatory Receptor (CCR) nucleic acid, comprising: (a) a first polynucleotide encoding an extracellular domain of Tumor Necrosis Factor Receptor 1 (TNFR1); (b) a second polynucleotide encoding a transmembrane domain polypeptide; and (c) a third polynucleotide encoding a cytosolic costimulatory signaling domain polypeptide.

33. The Chimeric Costimulatory Receptor (CCR) nucleic acid of claim 32, wherein the first polynucleotide encodes an oligopeptide at least 80%, at least 85%, at least 90%, at least 95%, at least 99% or 100% identical to SEQ ID NO: 1.

34. The Chimeric Costimulatory Receptor (CCR) nucleic acid of claim 32, wherein the third polynucleotide encodes a cytosolic costimulatory signaling domain from a Tumor Necrosis Factor Receptor Superfamily (TNFRSF) member.

35. The Chimeric Costimulatory Receptor (CCR) nucleic acid of claim 32, wherein the third polynucleotide encodes a cytosolic costimulatory signaling domain from 4-1BB, BAFFR, OX40, CD27, CD40, GITR, HVEM, OX40, RELT, TACI, TROY, or TWEAK.

36. The Chimeric Costimulatory Receptor (CCR) nucleic acid of claim 32, wherein the third polynucleotide encodes a cytosolic costimulatory signaling domain from BAFFR.

37. The Chimeric Costimulatory Receptor (CCR) nucleic acid of claim 32, wherein the third polynucleotide encodes a cytosolic costimulatory signaling domain from 4-1BB.

38. The Chimeric Costimulatory Receptor (CCR) nucleic acid of claim 32, wherein the third polynucleotide encodes a cytosolic costimulatory signaling domain from CD27.

39. The Chimeric Costimulatory Receptor (CCR) nucleic acid of claim 32, wherein the third polynucleotide encodes a cytosolic costimulatory signaling domain from HVEM.

40. The Chimeric Costimulatory Receptor (CCR) nucleic acid of claim 32, wherein the third polynucleotide encodes a cytosolic costimulatory signaling domain from OX40.

41. The Chimeric Costimulatory Receptor (CCR) nucleic acid of claim 32, wherein the third polynucleotide encodes a cytosolic costimulatory signaling domain from GITR.

42. The Chimeric Costimulatory Receptor (CCR) nucleic acid of claim 32, wherein the third polynucleotide encodes a cytosolic costimulatory signaling domain from TACI.

43. The Chimeric Costimulatory Receptor (CCR) nucleic acid of claim 32, wherein the third polynucleotide encodes an oligopeptide at least 80%, at least 85%, at least 90%, at least 95%, at least 99% or 100% identical to SEQ ID NO: 3.

44. The Chimeric Costimulatory Receptor (CCR) nucleic acid of claim 32, wherein the third polynucleotide encodes an oligopeptide at least 80%, at least 85%, at least 90%, at least 95%, at least 99% or 100% identical to SEQ ID NO: 4.

45. The Chimeric Costimulatory Receptor (CCR) nucleic acid of claim 32, wherein the transmembrane domain polypeptide is the transmembrane domain polypeptide from a member of the Tumor Necrosis Factor Receptor Superfamily.

46. The Chimeric Costimulatory Receptor (CCR) nucleic acid of claim 32, wherein the second polynucleotide encodes an oligopeptide at least 80%, at least 85%, at least 90%, at least 95%, at least 99% or 100% identical to SEQ ID NO: 5.

47. The Chimeric Costimulatory Receptor (CCR) nucleic acid of claim 32, wherein the second polynucleotide encodes an oligopeptide at least 80%, at least 85%, at least 90%, at least 95%, at least 99% or 100% identical to SEQ ID NO: 6.

48. The Chimeric Costimulatory Receptor (CCR) nucleic acid of claim 32, wherein the transmembrane domain and cytosolic costimulatory signaling domain are from the same costimulatory signaling protein.

49. The Chimeric Costimulatory Receptor (CCR) nucleic acid of claim 32, wherein the transmembrane and costimulatory signaling domains are derived from 4-1BB, BAFFR, OX40, CD27, CD40, GITR, HVEM, OX40, RELT, TACI, TROY, or TWEAK.

50. The Chimeric Costimulatory Receptor (CCR) nucleic acid of claim 32, wherein the first polynucleotide and second polynucleotide are joined directly and/or indirectly to the third polynucleotide.

51. The Chimeric Costimulatory Receptor (CCR) nucleic acid of claim 32, wherein the first polynucleotide and third polynucleotide are joined directly and/or indirectly to the second polynucleotide.

52. The Chimeric Costimulatory Receptor (CCR) nucleic acid of claim 32, wherein the first polynucleotide and/or the second polynucleotide are joined indirectly to the third polynucleotide by a linker.

53. The Chimeric Costimulatory Receptor (CCR) nucleic acid of claim 32, wherein the first polynucleotide and/or the third polynucleotide are joined indirectly to the second polynucleotide by a linker.

54. A Chimeric Costimulatory Receptor (CCR) nucleic acids, comprising: (a) a first polynucleotide encoding an extracellular domain of Tumor Necrosis Factor Receptor 2 (TNFR2); (b) a second polynucleotide encoding a transmembrane domain polypeptide; and (c) a third polynucleotide encoding a cytosolic costimulatory signaling domain polypeptide.

55. The Chimeric Costimulatory Receptor (CCR) nucleic acid of claim 54, wherein the third polynucleotide encodes a cytosolic costimulatory signaling domain from a Tumor Necrosis Factor Receptor Superfamily (TNFRSF) member.

56. The Chimeric Costimulatory Receptor (CCR) nucleic acid of claim 54, wherein the third polynucleotide encodes a cytosolic costimulatory signaling domain from 4-1BB, BAFFR, OX40, CD27, CD40, GITR, HVEM, OX40, RELT, TACI, TROY, or TWEAK.

57. The Chimeric Costimulatory Receptor (CCR) nucleic acid of claim 54, wherein the third polynucleotide encodes a cytosolic costimulatory signaling domain from BAFFR.

58. The Chimeric Costimulatory Receptor (CCR) nucleic acid of claim 54, wherein the third polynucleotide encodes a cytosolic costimulatory signaling domain from 4-1BB.

59. The Chimeric Costimulatory Receptor (CCR) nucleic acid of claim 54, wherein the third polynucleotide encodes a cytosolic costimulatory signaling domain from CD27.

60. The Chimeric Costimulatory Receptor (CCR) nucleic acid of claim 54, wherein the third polynucleotide encodes a cytosolic costimulatory signaling domain from HVEM.

61. The Chimeric Costimulatory Receptor (CCR) nucleic acid of claim 54, wherein the third polynucleotide encodes a cytosolic costimulatory signaling domain from OX40.

62. The Chimeric Costimulatory Receptor (CCR) nucleic acid of claim 54, wherein the third polynucleotide encodes a cytosolic costimulatory signaling domain from GITR.

63. The Chimeric Costimulatory Receptor (CCR) nucleic acid of claim 54, wherein the third polynucleotide encodes a cytosolic costimulatory signaling domain from TACI.

64. The Chimeric Costimulatory Receptor (CCR) nucleic acid of claim 54, wherein the third polynucleotide encodes an oligopeptide at least 80%, at least 85%, at least 90%, at least 95%, at least 99% or 100% identical to SEQ ID NO: 3.

65. The Chimeric Costimulatory Receptor (CCR) nucleic acid of claim 54, wherein the third polynucleotide encodes an oligopeptide at least 80%, at least 85%, at least 90%, at least 95%, at least 99% or 100% identical to SEQ ID NO: 4.

66. The Chimeric Costimulatory Receptor (CCR) nucleic acid of claim 54, wherein the transmembrane domain polypeptide is the transmembrane domain polypeptide from a member of the Tumor Necrosis Factor Receptor Superfamily.

67. The Chimeric Costimulatory Receptor (CCR) nucleic acid of claim 54, wherein the second polynucleotide encodes an oligopeptide at least 80%, at least 85%, at least 90%, at least 95%, at least 99% or 100% identical to SEQ ID NO: 5.

68. The Chimeric Costimulatory Receptor (CCR) nucleic acid of claim 54, wherein the second polynucleotide encodes an oligopeptide at least 80%, at least 85%, at least 90%, at least 95%, at least 99% or 100% identical to SEQ ID NO: 6.

69. The Chimeric Costimulatory Receptor (CCR) nucleic acid of claim 54, wherein the transmembrane domain and cytosolic costimulatory signaling domain are from the same costimulatory signaling protein.

70. The Chimeric Costimulatory Receptor (CCR) nucleic acid of claim 54, wherein the transmembrane and costimulatory signaling domains are derived from 4-1BB, BAFFR, OX40, CD27, CD40, GITR, HVEM, OX40, RELT, TACI, TROY, or TWEAK.

71. The Chimeric Costimulatory Receptor (CCR) nucleic acid of claim 54, wherein the first polynucleotide and second polynucleotide are joined directly and/or indirectly to the third polynucleotide.

72. The Chimeric Costimulatory Receptor (CCR) nucleic acid of claim 54, wherein the first polynucleotide and third polynucleotide are joined directly and/or indirectly to the second polynucleotide.

73. The Chimeric Costimulatory Receptor (CCR) nucleic acid of claim 54, wherein the first polynucleotide and/or the second polynucleotide are joined indirectly to the third polynucleotide by a linker.

74. The Chimeric Costimulatory Receptor (CCR) nucleic acid of claim 54, wherein the first polynucleotide and/or the third polynucleotide are joined indirectly to the second polynucleotide by a linker.

75. A Chimeric Costimulatory Receptor (CCR) nucleic acid, comprising: (a) a first polynucleotide encoding an extracellular domain of Tumor Necrosis Factor Receptor Superfamily Member 6 (TNFRSF6; Fas); (b) a second polynucleotide encoding a transmembrane domain polypeptide; and (c) a third polynucleotide encoding a cytosolic costimulatory signaling domain polypeptide.

76. The Chimeric Costimulatory Receptor (CCR) nucleic acid of claim 75, wherein the first polynucleotide encodes an oligopeptide at least 80%, at least 85%, at least 90%, at least 95%, at least 99% or 100% identical to SEQ ID NO: 2.

77. The Chimeric Costimulatory Receptor (CCR) nucleic acid of claim 75, wherein the third polynucleotide encodes a cytosolic costimulatory signaling domain from a Tumor Necrosis Factor Receptor Superfamily (TNFRSF) member.

78. The Chimeric Costimulatory Receptor (CCR) nucleic acid of claim 75, wherein the third polynucleotide encodes a cytosolic costimulatory signaling domain from 4-1BB, BAFFR, OX40, CD27, CD40, GITR, HVEM, OX40, RELT, TACI, TROY, or TWEAK.

79. The Chimeric Costimulatory Receptor (CCR) nucleic acid of claim 75, wherein the third polynucleotide encodes a cytosolic costimulatory signaling domain from BAFFR.

80. The Chimeric Costimulatory Receptor (CCR) nucleic acid of claim 75, wherein the third polynucleotide encodes a cytosolic costimulatory signaling domain from 4-1BB.

81. The Chimeric Costimulatory Receptor (CCR) nucleic acid of claim 75, wherein the third polynucleotide encodes a cytosolic costimulatory signaling domain from CD27.

82. The Chimeric Costimulatory Receptor (CCR) nucleic acid of claim 75, wherein the third polynucleotide encodes a cytosolic costimulatory signaling domain from HVEM.

83. The Chimeric Costimulatory Receptor (CCR) nucleic acid of claim 75, wherein the third polynucleotide encodes a cytosolic costimulatory signaling domain from OX40.

84. The Chimeric Costimulatory Receptor (CCR) nucleic acid of claim 75, wherein the third polynucleotide encodes a cytosolic costimulatory signaling domain from GITR.

85. The Chimeric Costimulatory Receptor (CCR) nucleic acid of claim 75, wherein the third polynucleotide encodes a cytosolic costimulatory signaling domain from TACI.

86. The Chimeric Costimulatory Receptor (CCR) nucleic acid of claim 75, wherein the third polynucleotide encodes an oligopeptide at least 80%, at least 85%, at least 90%, at least 95%, at least 99% or 100% identical to SEQ ID NO: 3.

87. The Chimeric Costimulatory Receptor (CCR) nucleic acid of claim 75, wherein the third polynucleotide encodes an oligopeptide at least 80%, at least 85%, at least 90%, at least 95%, at least 99% or 100% identical to SEQ ID NO: 4.

88. The Chimeric Costimulatory Receptor (CCR) nucleic acid of claim 75, wherein the transmembrane domain polypeptide is the transmembrane domain polypeptide from a member of the Tumor Necrosis Factor Receptor Superfamily.

89. The Chimeric Costimulatory Receptor (CCR) nucleic acid of claim 75, wherein the second polynucleotide encodes an oligopeptide at least 80%, at least 85%, at least 90%, at least 95%, at least 99% or 100% identical to SEQ ID NO: 5.

90. The Chimeric Costimulatory Receptor (CCR) nucleic acid of claim 75, wherein the second polynucleotide encodes an oligopeptide at least 80%, at least 85%, at least 90%, at least 95%, at least 99% or 100% identical to SEQ ID NO: 6.

91. The Chimeric Costimulatory Receptor (CCR) nucleic acid of claim 75, wherein the transmembrane domain and cytosolic costimulatory signaling domain are from the same costimulatory signaling protein.

92. The Chimeric Costimulatory Receptor (CCR) nucleic acid of claim 75, wherein the transmembrane and costimulatory signaling domains are derived from 4-1BB, BAFFR, OX40, CD27, CD40, GITR, HVEM, OX40, RELT, TACI, TROY, or TWEAK.

93. The Chimeric Costimulatory Receptor (CCR) nucleic acid of claim 75, wherein the first polynucleotide and second polynucleotide are joined directly and/or indirectly to the third polynucleotide.

94. The Chimeric Costimulatory Receptor (CCR) nucleic acid of claim 75, wherein the first polynucleotide and third polynucleotide are joined directly and/or indirectly to the second polynucleotide.

95. The Chimeric Costimulatory Receptor (CCR) nucleic acid of claim 75, wherein the first polynucleotide and/or the second polynucleotide are joined indirectly to the third polynucleotide by a linker.

96. The Chimeric Costimulatory Receptor (CCR) nucleic acid of claim 75, wherein the first polynucleotide and/or the third polynucleotide are joined indirectly to the second polynucleotide by a linker.

97. A Chimeric Costimulatory Receptor (CCR) polypeptide, comprising (a) a first oligopeptide comprising extracellular domain of a member of the Tumor Necrosis Factor Receptor Superfamily; (b) a second oligopeptide comprising a transmembrane domain polypeptide; and (c) a third oligopeptide comprising a cytosolic costimulatory signaling domain polypeptide.

98. The Chimeric Costimulatory Receptor (CCR) polypeptide of claim 97, wherein the first oligopeptide and the third oligopeptide are derived from different members of the TNFRSF.

99. The Chimeric Costimulatory Receptor (CCR) polypeptide of claim 97, wherein the first oligopeptide comprises an extracellular domain of TNFRSF member having a death domain.

100. The Chimeric Costimulatory Receptor (CCR) polypeptide of claim 97, wherein the first oligopeptide comprises an extracellular domain of TNFR1, TNFR2, Fas, DR4, DR5, DR3, DR6, EDAR, XEDAR, TROY or NGFR.

101. The Chimeric Costimulatory Receptor (CCR) polypeptide of claim 97, wherein the first oligopeptide comprises an extracellular domain of TNFR1.

102. The Chimeric Costimulatory Receptor (CCR) polypeptide of claim 97, wherein the first oligopeptide comprises an extracellular domain of TNFR2.

103. The Chimeric Costimulatory Receptor (CCR) polypeptide of claim 97, wherein the first oligopeptide comprises an extracellular domain of Fas.

104. The Chimeric Costimulatory Receptor (CCR) polypeptide of claim 97, wherein the first oligopeptide comprises an extracellular domain of DR4.

105. The Chimeric Costimulatory Receptor (CCR) polypeptide of claim 97, wherein the first oligopeptide comprises an extracellular domain of DR5.

106. The Chimeric Costimulatory Receptor (CCR) polypeptide of claim 97, wherein the first oligopeptide comprises a sequence at least 80%, at least 85%, at least 90%, at least 95%, at least 99% or 100% identical to SEQ ID NO: 1.

107. The Chimeric Costimulatory Receptor (CCR) nucleic acid of claim 97, wherein the first oligopeptide comprises a sequence at least 80%, at least 85%, at least 90%, at least 95%, at least 99% or 100% identical to SEQ ID NO: 2.

108. The Chimeric Costimulatory Receptor (CCR) polypeptide of claim 97, wherein the third oligopeptide comprises a cytosolic costimulatory signaling domain from a Tumor Necrosis Factor Receptor Superfamily (TNFRSF) member.

109. The Chimeric Costimulatory Receptor (CCR) polypeptide of claim 97, wherein the third oligopeptide comprises a cytosolic costimulatory signaling domain from 4-1BB, BAFFR, OX40, CD27, CD40, GITR, HVEM, OX40, RELT, TACI, TROY, or TWEAK.

110. The Chimeric Costimulatory Receptor (CCR) polypeptide of claim 97, wherein the third oligopeptide comprises a cytosolic costimulatory signaling domain from BAFFR.

111. The Chimeric Costimulatory Receptor (CCR) polypeptide of claim 97, wherein the third oligopeptide comprises a cytosolic costimulatory signaling domain from 4-1BB.

112. The Chimeric Costimulatory Receptor (CCR) polypeptide of claim 97, wherein the third oligopeptide comprises a cytosolic costimulatory signaling domain from CD27.

113. The Chimeric Costimulatory Receptor (CCR) polypeptide of claim 97, wherein the third oligopeptide comprises a cytosolic costimulatory signaling domain from HVEM.

114. The Chimeric Costimulatory Receptor (CCR) polypeptide of claim 97, wherein the third oligopeptide comprises a cytosolic costimulatory signaling domain from OX40.

115. The Chimeric Costimulatory Receptor (CCR) polypeptide of claim 97, wherein the third oligopeptide comprises a cytosolic costimulatory signaling domain from GITR.

116. The Chimeric Costimulatory Receptor (CCR) polypeptide of claim 97, wherein the third oligopeptide comprises a cytosolic costimulatory signaling domain from TACI.

117. The Chimeric Costimulatory Receptor (CCR) polypeptide of claim 97, wherein the third oligopeptide comprises a sequence at least 80%, at least 85%, at least 90%, at least 95%, at least 99% or 100% identical to SEQ ID NO: 3.

118. The Chimeric Costimulatory Receptor (CCR) polypeptide of claim 97, wherein the third oligopeptide comprises a sequence at least 80%, at least 85%, at least 90%, at least 95%, at least 99% or 100% identical to SEQ ID NO: 4.

119. The Chimeric Costimulatory Receptor (CCR) polypeptide of claim 97, wherein the transmembrane domain polypeptide is the transmembrane domain polypeptide from a member of the Tumor Necrosis Factor Receptor Superfamily.

120. The Chimeric Costimulatory Receptor (CCR) polypeptide of claim 97, wherein the second oligopeptide comprises a sequence at least 80%, at least 85%, at least 90%, at least 95%, at least 99% or 100% identical to SEQ ID NO: 5.

121. The Chimeric Costimulatory Receptor (CCR) nucleic acid of claim 97, wherein the second oligopeptide comprises a sequence at least 80%, at least 85%, at least 90%, at least 95%, at least 99% or 100% identical to SEQ ID NO: 6.

122. The Chimeric Costimulatory Receptor (CCR) polypeptide of claim 97, wherein the transmembrane domain and cytosolic costimulatory signaling domain are from the same costimulatory signaling protein.

123. The Chimeric Costimulatory Receptor (CCR) polypeptide of claim 97, wherein the transmembrane and costimulatory signaling domains are derived from 4-1BB, BAFFR, OX40, CD27, CD40, GITR, HVEM, OX40, RELT, TACI, TROY, or TWEAK.

124. The Chimeric Costimulatory Receptor (CCR) polypeptide of claim 97, wherein the first oligopeptide and second oligopeptide are joined directly and/or indirectly to the third polynucleotide.

125. The Chimeric Costimulatory Receptor (CCR) polypeptide of claim 97, wherein the first oligopeptide and third oligopeptide are joined directly and/or indirectly to the second oligopeptide.

126. The Chimeric Costimulatory Receptor (CCR) polypeptide of claim 97, wherein the first oligopeptide and/or the second oligopeptide are joined indirectly to the third oligopeptide by a linker.

127. The Chimeric Costimulatory Receptor (CCR) polypeptide of claim 97, wherein the first oligopeptide and/or the third oligopeptide are joined indirectly to the second oligopeptide by a linker.

128. A Chimeric Costimulatory Receptor (CCR) polypeptide, comprising: (a) a first oligopeptide comprising an extracellular domain of Tumor Necrosis Factor Receptor 1 (TNFR1); (b) a second oligopeptide comprising a transmembrane domain polypeptide; and (c) a third oligopeptide comprising a cytosolic costimulatory signaling domain polypeptide.

129. The Chimeric Costimulatory Receptor (CCR) polypeptide of claim 128, wherein the first oligopeptide comprises a sequence at least 80%, at least 85%, at least 90%, at least 95%, at least 99% or 100% identical to SEQ ID NO: 1.

130. The Chimeric Costimulatory Receptor (CCR) polypeptide of claim 128, wherein the third oligopeptide comprises a cytosolic costimulatory signaling domain from a Tumor Necrosis Factor Receptor Superfamily (TNFRSF) member.

131. The Chimeric Costimulatory Receptor (CCR) polypeptide of claim 128, wherein the third oligopeptide comprises a cytosolic costimulatory signaling domain from 4-1BB, BAFFR, OX40, CD27, CD40, GITR, HVEM, OX40, RELT, TACI, TROY, or TWEAK.

132. The Chimeric Costimulatory Receptor (CCR) polypeptide of claim 128, wherein the third oligopeptide comprises a cytosolic costimulatory signaling domain from BAFFR.

133. The Chimeric Costimulatory Receptor (CCR) polypeptide of claim 128, wherein the third oligopeptide comprises a cytosolic costimulatory signaling domain from 4-1BB.

134. The Chimeric Costimulatory Receptor (CCR) polypeptide of claim 128, wherein the third oligopeptide comprises a cytosolic costimulatory signaling domain from CD27.

135. The Chimeric Costimulatory Receptor (CCR) polypeptide of claim 128, wherein the third oligopeptide comprises a cytosolic costimulatory signaling domain from HVEM.

136. The Chimeric Costimulatory Receptor (CCR) polypeptide of claim 128, wherein the third oligopeptide comprises a cytosolic costimulatory signaling domain from OX40.

137. The Chimeric Costimulatory Receptor (CCR) polypeptide of claim 128, wherein the third oligopeptide comprises a cytosolic costimulatory signaling domain from GITR.

138. The Chimeric Costimulatory Receptor (CCR) polypeptide of claim 128, wherein the third oligopeptide comprises a cytosolic costimulatory signaling domain from TACI.

139. The Chimeric Costimulatory Receptor (CCR) polypeptide of claim 128, wherein the third oligopeptide comprises a sequence at least 80%, at least 85%, at least 90%, at least 95%, at least 99% or 100% identical to SEQ ID NO: 3.

140. The Chimeric Costimulatory Receptor (CCR) polypeptide of claim 128, wherein the third oligopeptide comprises a sequence at least 80%, at least 85%, at least 90%, at least 95%, at least 99% or 100% identical to SEQ ID NO: 4.

141. The Chimeric Costimulatory Receptor (CCR) polypeptide of claim 128, wherein the transmembrane domain polypeptide is the transmembrane domain polypeptide from a member of the Tumor Necrosis Factor Receptor Superfamily.

142. The Chimeric Costimulatory Receptor (CCR) polypeptide of claim 128, wherein the second oligopeptide comprises a sequence at least 80%, at least 85%, at least 90%, at least 95%, at least 99% or 100% identical to SEQ ID NO: 5.

143. The Chimeric Costimulatory Receptor (CCR) nucleic acid of claim 128, wherein the second oligopeptide comprises a sequence at least 80%, at least 85%, at least 90%, at least 95%, at least 99% or 100% identical to SEQ ID NO: 6.

144. The Chimeric Costimulatory Receptor (CCR) polypeptide of claim 128, wherein the transmembrane domain and cytosolic costimulatory signaling domain are from the same costimulatory signaling protein.

145. The Chimeric Costimulatory Receptor (CCR) polypeptide of claim 128, wherein the transmembrane and costimulatory signaling domains are derived from 4-1BB, BAFFR, OX40, CD27, CD40, GITR, HVEM, OX40, RELT, TACI, TROY, or TWEAK.

146. The Chimeric Costimulatory Receptor (CCR) polypeptide of claim 128, wherein the first oligopeptide and second oligopeptide are joined directly and/or indirectly to the third oligopeptide.

147. The Chimeric Costimulatory Receptor (CCR) polypeptide of claim 128, wherein the first oligopeptide and third oligopeptide are joined directly and/or indirectly to the second oligopeptide.

148. The Chimeric Costimulatory Receptor (CCR) polypeptide of claim 128, wherein the first oligopeptide and/or the second oligopeptide are joined indirectly to the third oligopeptide by a linker.

149. The Chimeric Costimulatory Receptor (CCR) polypeptide of claim 128, wherein the first oligopeptide and/or the third oligopeptide are joined indirectly to the second oligopeptide by a linker.

150. A Chimeric Costimulatory Receptor (CCR) polypeptide, comprising: (a) a first oligopeptide comprising an extracellular domain of Tumor Necrosis Factor Receptor 2 (TNFR2); (b) a second oligopeptide comprising a transmembrane domain polypeptide; and (c) a third oligopeptide comprising a cytosolic costimulatory signaling domain polypeptide.

151. The Chimeric Costimulatory Receptor (CCR) polypeptide of claim 150, wherein the third oligopeptide comprises a cytosolic costimulatory signaling domain from a Tumor Necrosis Factor Receptor Superfamily (TNFRSF) member.

152. The Chimeric Costimulatory Receptor (CCR) polypeptide of claim 150, wherein the third oligopeptide comprises a cytosolic costimulatory signaling domain from 4-1BB, BAFFR, OX40, CD27, CD40, GITR, HVEM, OX40, RELT, TACI, TROY, or TWEAK.

153. The Chimeric Costimulatory Receptor (CCR) polypeptide of claim 150, wherein the third oligopeptide comprises a cytosolic costimulatory signaling domain from BAFFR.

154. The Chimeric Costimulatory Receptor (CCR) polypeptide of claim 150, wherein the third oligopeptide comprises a cytosolic costimulatory signaling domain from 4-1BB.

155. The Chimeric Costimulatory Receptor (CCR) polypeptide of claim 150, wherein the third oligopeptide comprises a cytosolic costimulatory signaling domain from CD27.

156. The Chimeric Costimulatory Receptor (CCR) polypeptide of claim 150, wherein the third oligopeptide comprises a cytosolic costimulatory signaling domain from HVEM.

157. The Chimeric Costimulatory Receptor (CCR) polypeptide of claim 150, wherein the third oligopeptide comprises a cytosolic costimulatory signaling domain from OX40.

158. The Chimeric Costimulatory Receptor (CCR) polypeptide of claim 150, wherein the third oligopeptide comprises a cytosolic costimulatory signaling domain from GITR.

159. The Chimeric Costimulatory Receptor (CCR) polypeptide of claim 150, wherein the third oligopeptide comprises a cytosolic costimulatory signaling domain from TACI.

160. The Chimeric Costimulatory Receptor (CCR) polypeptide of claim 150, wherein the third oligopeptide comprises a sequence at least 80%, at least 85%, at least 90%, at least 95%, at least 99% or 100% identical to SEQ ID NO: 3.

161. The Chimeric Costimulatory Receptor (CCR) polypeptide of claim 150, wherein the third oligopeptide comprises a sequence at least 80%, at least 85%, at least 90%, at least 95%, at least 99% or 100% identical to SEQ ID NO: 4.

162. The Chimeric Costimulatory Receptor (CCR) polypeptide of claim 150, wherein the transmembrane domain polypeptide is the transmembrane domain polypeptide from a member of the Tumor Necrosis Factor Receptor Superfamily.

163. The Chimeric Costimulatory Receptor (CCR) polypeptide of claim 150, wherein the second oligopeptide comprises a sequence at least 80%, at least 85%, at least 90%, at least 95%, at least 99% or 100% identical to SEQ ID NO: 5.

164. The Chimeric Costimulatory Receptor (CCR) nucleic acid of claim 150, wherein the second oligopeptide comprises a sequence at least 80%, at least 85%, at least 90%, at least 95%, at least 99% or 100% identical to SEQ ID NO: 6.

165. The Chimeric Costimulatory Receptor (CCR) polypeptide of claim 150, wherein the transmembrane domain and cytosolic costimulatory signaling domain are from the same costimulatory signaling protein.

166. The Chimeric Costimulatory Receptor (CCR) polypeptide of claim 150, wherein the transmembrane and costimulatory signaling domains are derived from 4-1BB, BAFFR, OX40, CD27, CD40, GITR, HVEM, OX40, RELT, TACI, TROY, or TWEAK.

167. The Chimeric Costimulatory Receptor (CCR) polypeptide of claim 150, wherein the first oligopeptide and second oligopeptide are joined directly and/or indirectly to the third oligopeptide.

168. The Chimeric Costimulatory Receptor (CCR) polypeptide of claim 150, wherein the first oligopeptide and third oligopeptide are joined directly and/or indirectly to the second oligopeptide.

169. The Chimeric Costimulatory Receptor (CCR) polypeptide of claim 150, wherein the first oligopeptide and/or the second oligopeptide are joined indirectly to the third oligopeptide by a linker.

170. The Chimeric Costimulatory Receptor (CCR) polypeptide of claim 150, wherein the first oligopeptide and/or the third oligopeptide are joined indirectly to the second oligopeptide by a linker.

171. A Chimeric Costimulatory Receptor (CCR) polypeptide, comprising: (a) a first oligopeptide comprising an extracellular domain of Tumor Necrosis Factor Receptor Superfamily Member 6 (TNFRSF6; Fas); (b) a second oligopeptide comprising a transmembrane domain polypeptide; and (c) a third oligopeptide comprising a cytosolic costimulatory signaling domain polypeptide.

172. The Chimeric Costimulatory Receptor (CCR) nucleic acid of claim 171, wherein the first oligopeptide comprises a sequence at least 80%, at least 85%, at least 90%, at least 95%, at least 99% or 100% identical to SEQ ID NO: 2.

173. The Chimeric Costimulatory Receptor (CCR) polypeptide of claim 171, wherein the third oligopeptide comprises a cytosolic costimulatory signaling domain from a Tumor Necrosis Factor Receptor Superfamily (TNFRSF) member.

174. The Chimeric Costimulatory Receptor (CCR) polypeptide of claim 171, wherein the third oligopeptide comprises a cytosolic costimulatory signaling domain from 4-1BB, BAFFR, OX40, CD27, CD40, GITR, HVEM, OX40, RELT, TACI, TROY, or TWEAK.

175. The Chimeric Costimulatory Receptor (CCR) polypeptide of claim 171, wherein the third oligopeptide comprises a cytosolic costimulatory signaling domain from BAFFR.

176. The Chimeric Costimulatory Receptor (CCR) polypeptide of claim 171, wherein the third oligopeptide comprises a cytosolic costimulatory signaling domain from 4-1BB.

177. The Chimeric Costimulatory Receptor (CCR) polypeptide of claim 171, wherein the third oligopeptide comprises a cytosolic costimulatory signaling domain from CD27.

178. The Chimeric Costimulatory Receptor (CCR) polypeptide of claim 171, wherein the third oligopeptide comprises a cytosolic costimulatory signaling domain from HVEM.

179. The Chimeric Costimulatory Receptor (CCR) polypeptide of claim 171, wherein the third oligopeptide comprises a cytosolic costimulatory signaling domain from OX40.

180. The Chimeric Costimulatory Receptor (CCR) polypeptide of claim 171, wherein the third oligopeptide comprises a cytosolic costimulatory signaling domain from GITR.

181. The Chimeric Costimulatory Receptor (CCR) polypeptide of claim 171, wherein the third oligopeptide comprises a cytosolic costimulatory signaling domain from TACI.

182. The Chimeric Costimulatory Receptor (CCR) polypeptide of claim 171, wherein the third oligopeptide comprises a sequence at least 80%, at least 85%, at least 90%, at least 95%, at least 99% or 100% identical to SEQ ID NO: 3.

183. The Chimeric Costimulatory Receptor (CCR) polypeptide of claim 171, wherein the third oligopeptide comprises a sequence at least 80%, at least 85%, at least 90%, at least 95%, at least 99% or 100% identical to SEQ ID NO: 4.

184. The Chimeric Costimulatory Receptor (CCR) polypeptide of claim 171, wherein the transmembrane domain polypeptide is the transmembrane domain polypeptide from a member of the Tumor Necrosis Factor Receptor Superfamily.

185. The Chimeric Costimulatory Receptor (CCR) polypeptide of claim 171, wherein the second oligopeptide comprises a sequence at least 80%, at least 85%, at least 90%, at least 95%, at least 99% or 100% identical to SEQ ID NO: 5.

186. The Chimeric Costimulatory Receptor (CCR) nucleic acid of claim 171, wherein the second oligopeptide comprises a sequence at least 80%, at least 85%, at least 90%, at least 95%, at least 99% or 100% identical to SEQ ID NO: 6.

187. The Chimeric Costimulatory Receptor (CCR) polypeptide of claim 171, wherein the transmembrane domain and cytosolic costimulatory signaling domain are from the same costimulatory signaling protein.

188. The Chimeric Costimulatory Receptor (CCR) polypeptide of claim 171, wherein the transmembrane and costimulatory signaling domains are derived from 4-1BB, BAFFR, OX40, CD27, CD40, GITR, HVEM, OX40, RELT, TACI, TROY, or TWEAK.

189. The Chimeric Costimulatory Receptor (CCR) polypeptide of claim 171, wherein the first oligopeptide and second oligopeptide are joined directly and/or indirectly to the third oligopeptide.

190. The Chimeric Costimulatory Receptor (CCR) polypeptide of claim 171, wherein the first oligopeptide and third oligopeptide are joined directly and/or indirectly to the second oligopeptide.

191. The Chimeric Costimulatory Receptor (CCR) polypeptide of claim 171, wherein the first oligopeptide and/or the second oligopeptide are joined indirectly to the third oligopeptide by a linker.

192. The Chimeric Costimulatory Receptor (CCR) polypeptide of claim 171, wherein the first oligopeptide and/or the third oligopeptide are joined indirectly to the second oligopeptide by a linker.

193. A Chimeric Costimulatory Receptor (CCR) polypeptide at least 80%, at least 85%, at least 90%, at least 95%, at least 99% or 100% identical to SEQ ID NO: 7.

194. A Chimeric Costimulatory Receptor (CCR) polypeptide at least 80%, at least 85%, at least 90%, at least 95%, at least 99% or 100% identical to SEQ ID NO: 8.

195. A Chimeric Costimulatory Receptor (CCR) polypeptide at least 80%, at least 85%, at least 90%, at least 95%, at least 99% or 100% identical to SEQ ID NO: 9.

196. A Chimeric Costimulatory Receptor (CCR) polypeptide at least 80%, at least 85%, at least 90%, at least 95%, at least 99% or 100% identical to SEQ ID NO: 10.

197. A T-cell, comprising a nucleic acid according to any one of claims 1-96.

198. The T cell according to claim 197, wherein the T cell is a cytotoxic T cell, helper T cell, regulatory T cell, or gamma-delta T cell.

199. The T-cell of claim 197, further comprising a second nucleic acid encoding an engineered T cell receptor (TCR) or a synthetic antigen receptor polypeptide that can recognize a target-specific ligand.

200. The T-cell of claim 197, wherein the target-specific ligand binds an antigen on a cancerous cell.

201. The T-cell of claim 197, wherein the synthetic antigen receptor polynucleotide is a Chimeric Antigen Receptor (CAR), a T cell Antigen Coupler (TAC), or a BiTE.

202. The T-cell of claim 197, wherein the synthetic antigen receptor polynucleotide is a T cell Antigen Coupler (TAC).

203. A T-cell, comprising a polypeptide according to anyone of claims 97-194.

204. The T cell according to claim 203, wherein the T cell is a cytotoxic T cell, helper T cell, regulatory T cell, or gamma-delta T cell.

205. The T-cell of claim 203, further comprising an engineered T cell receptor (TCR) or a synthetic antigen receptor polypeptide that can recognize a target-specific ligand.

206. The T-cell of claim 203, wherein the target-specific ligand binds an antigen on a cancerous cell.

207. The T-cell of claim 203, wherein the synthetic antigen receptor polypeptide is a Chimeric Antigen Receptor (CAR), a T cell Antigen Coupler (TAC), or a BiTE.

208. The T-cell of claim 203, wherein the synthetic antigen receptor polypeptide is a T cell Antigen Coupler (TAC).

209. A method of treating a cancer in an individual in need thereof, comprising administering to the individual a T-cell according to any one of claims 197-208.

210. The method of claim 209, wherein the cancer is a leukemia or lymphoma.

211. The method of claim 209, wherein the cancer is mixed lineage leukemia (MLL), chronic lymphocytic leukemia (CLL), acute lymphoblastic leukemia (ALL), large B-cell lymphoma, diffuse large B-cell lymphoma, primary mediastinal B cell lymphoma, high grade B-cell lymphoma, or large B cell lymphoma arising from follicular lymphoma.

212. The method of claim 209, wherein the cancer is a lung cancer, a breast cancer, a colon cancer, multiple myeloma, glioblastoma, gastric cancer, ovarian cancer, stomach cancer, colorectal cancer, urothelial cancer, endometrial cancer, or a melanoma.

213. The method of claim 209, wherein the cancer is a lung cancer.

214. The method of claim 209, wherein the cancer is a breast cancer.

215. The method of claim 209, wherein the cancer is a colon cancer.

216. The method of claim 209, wherein the cancer is multiple myeloma.

217. The method of claim 209, wherein the cancer is a glioblastoma.

218. The method of claim 209, wherein the cancer is a gastric cancer.

219. The method of claim 209, wherein the cancer is an ovarian cancer.

220. The method of claim 209, wherein the cancer is a stomach cancer.

221. The method of claim 209, wherein the cancer is a colorectal cancer.

222. The method of claim 209, wherein the cancer is urothelial cancer.

223. The method of claim 209, wherein the cancer is an endometrial cancer.

224. The method of claim 209, wherein the cancer is a melanoma.

225. A pharmaceutical composition, comprising (a) T-cell according to anyone of claims 197-208; and (b) a pharmaceutically acceptable carrier.

226. A vector construct comprising: (a) a Chimeric Costimulatory Receptor (CCR) nucleic acid according to any one of claims 1-96; and (b) a promoter functional in a mammalian cell.
